(19) United States
(12) Patent Application Publication
(10) Pub. No.: US 2010/0168210 A1
(43) Pub. Date: Jul. 1, 2010

(54) T-Cell Cytokine-Inducing Surface Molecules and Methods of Use

(76) Inventors: Carl Keith Edwards, III, Commerce City, CO (US); Li Li, Centennial, CO (US); Karen R. Jonscher, Centennial, CO (US); David Norris, Greenwood Village, CO (US)

Correspondence Address: Don D. Cha, 547 Buena Vista Road, Golden, CO, 80401, US

(21) Appl. No.: 12/663,328

(22) PCT Filed: Jun. 5, 2008
(86) PCT No.: PCT/US08/65992

§ 371 Date: Dec. 7, 2009

Related U.S. Application Data
(60) Provisional application No. 60/942,041, filed on Jun. 5, 2007.

(51) Int. Cl. A61K 31/7088; A61P 19/02; A61P 35/00; A61P 17/06; A61P 3/10; A61P 11/06; A61P 31/18; A61P 31/12

(52) U.S. Cl. 514/44 A

(57) ABSTRACT

The invention provides cytokine modulators and methods for using the same to modulate cytokine production in monocyte lineage-derived cells. In particular, cytokine modulators of the invention selectively bind to a T-cell cytokine-inducing surface molecule (TCISM)-ligand of T lymphocytes or the corresponding TCISM-receptor of monocyte lineage-derived cells, thereby modulating cytokine production in monocyte lineage-derived cells.

### FIELD OF THE INVENTION

The invention relates to cytokine modulators and methods for using the same to modulate cytokine production in monocyte lineage-derived cells.

### BACKGROUND OF THE INVENTION

A wide variety of clinical conditions are mediated by acute and/or chronic inflammation including, but not limited to, Rheumatoid Arthritis, Multiple Sclerosis, Crohn's Disease, Psoriasis, Psoriatic Arthritis, Graves Disease, Autoimmune Polyendocrine Syndromes, Hereditary Proteinuria Syndrome, Type I Diabetes, Systemic Lupus Erythematosus, Primary Bilary Cirrhosis, Autoimmune Thyroiditis, Hepatitis, Acquired Immunodeficiency Disease (HIV), Graft versus Host Disease, Allograft Disease, Asthma, Cutaneous T-Cell Lymphoma, HTLV-I-Associated Cutaneous T-Cell Lymhoma, HTLV-II-Associated Lymphoma, Hairy Cell Leukemia, Idiopathic CD4+ T-Lymphocytopenia, or Melanoma. It is believed that one of the mechanisms involved in inflammation is up-regulation of cytokines by activating monocyte lineage-derived macrophages (Mφ). For example, proinflammatory cytokines such as Tumor Necrosis Factor-α (TNFα) and Interleukin-1β (IL-1β), produced in the lesion, have been shown to induce and maintain chronic lesional inflammation. Recent studies in relevant animal models suggest that T-cells (T_{c}) play a key role in Mφ activation; however, T-cell cytokines, such as Interleukin's-4, 10, and 13 (IL-4, IL-10 and IL-13), have been shown to either play an anti-inflammatory role or only weakly induce TNFα/IL-1β up-regulation.

Therefore, there is a need to modulate inflammation to treat various clinical conditions associated with acute and/or chronic inflammation.

### SUMMARY OF THE INVENTION

Some aspects of the invention relate to cytokine modulators and methods for using the same to modulate cytokine production in monocyte lineage-derived cells. In some particular embodiments, the invention provides proinflammatory cytokine modulators and methods for using the same to modulate proinflammatory cytokine production in monocyte lineage-derived cells, typically human monocyte lineage-derived cells. Without being bound by any theory, it is believed that monocyte lineage-derived cells become activated once they come in direct cell-cell contact with different effector T cell populations. Controlling adaptive (i.e., acquired) immunity at the level of TCISM Ligand and/or TCISM Receptor provides therapeutic intervention that still allow for innate immunity during microorganism infections (e.g., bacterial skin infections).

One aspect of the invention provides a method for modulating cytokine production in monocyte lineage-derived cells of a subject comprising administering a cytokine modulator to said subject, wherein the cytokine modulator selectively binds to a T-cell cytokine-inducing surface molecule (TCISM)-ligand of T lymphocytes or the corresponding TCISM-receptor of monocyte lineage-derived cells, whereby selective binding of the cytokine modulator to the TCISM-ligand or the TCISM-receptor modulates cytokine production in monocyte lineage-derived cells.

In some embodiments, TCISM-ligand comprises at least one of the TCISM-ligand listed in Table 1 (FIG. 19). Within these embodiments, in some instances the TCISM-ligand comprises CD81, CD21, CD316, α-Enolase, FKBP4, other members of the FKBP Multigene Family, or a combination thereof. Members of the FKBP Multigene Family include, but are not limited to, FKBP12 (FKBP1A), FKBP12.6 (FKBP1B), FKBP13 (FKBP2), FKBP9 (FKBP11), FKBP22 (FKBP14), FKBP23 (FKBP7), FKBP25 (FKBP3), FKBP36 (FKBP6), FKBP37 (AIP), FKBP38 (FKBP8), FKBP51 (FKBP5), FKBP52 (FKBP4), FKBP60 (FKBP9), and FKBP65 (FKBP10).

In other embodiments, monocyte lineage-derived cells comprise monocyte lineage-derived macrophages, antigen-presenting cells (APC), dendritic cells, Langerhans cells, Kuppfler Cells, or a combination thereof.

Still in other embodiments, T lymphocytes are CD3⁺ T lymphocytes.

Yet in other embodiments, the modulated cytokine comprises Tumor Necrosis Factor-α (TNF-α), Interleukin-1β (IL-1β), Interleukin-32 (IL-32), or a combination thereof.

In other embodiments, TCISM-ligand is a TCISM-ligand that is present on CD3⁺ lymphocytes. Within these embodiments, in some cases TCISM-ligand comprises CD81, CD21, CD315, CD316, α-enolase, a FKBP, or a combination thereof. Exemplary FKBPs include, but are not limited to, FKBP4, FKBP12 (FKBP1A), FKBP12.6 (FKBP1B), FKBP13 (FKBP2), FKBP9 (FKBP11), FKBP22 (FKBP14), FKBP23 (FKBP7), FKBP25 (FKBP3), FKBP36 (FKBP6), FKBP37 (AIP), FKBP38 (FKBP8), FKBP51 (FKBP5), FKBP52 (FKBP4), FKBP60 (FKBP9), and FKBP65 (FKBP10).

Still in other embodiments, the TCISM-receptor comprises a TCISM-receptor that is present on CD68⁺ antigen-presenting cells. Within these embodiments, in some instances the TCISM-receptor comprises a receptor for CD81, a receptor for CD21, a receptor for CD315, a receptor for CD316, a receptor for α-enolase, a receptor for FK binding protein, or a combination thereof. In some cases, the TCISM-receptor comprises a receptor for CD19, CD21, CD225, CD315, CD316, C3dR, CD19, CD81, BCR, CD9, CD81, KAI1/CD82, FK506, Rapamycin (Sirolimus), Everolimus, Cyclosporin, Tacrolimus, other synthetic small molecule immunosuppressant agents, or a combination thereof. In general, the TCISM-receptor refers to a receptor that is present on monocyte lineage-derived cells which, when bound to a ligand, stimulates cytokine production in monocyte lineage-derived cells.

Another aspect of the invention provides a method for treating a clinical condition mediated by acute or chronic inflammation in a subject comprising administering a cytokine modulator to said subject, wherein the cytokine modulator selectively binds to a T-cell cytokine-inducing surface molecule (TCISM)-ligand of T lymphocytes or the corresponding TCISM-receptor of monocyte lineage-derived cells, whereby modulation of cytokine production by the cytokine modulator is used to treat the clinical condition mediated by acute or chronic inflammation.

In some embodiments, the cytokine modulator binds selectively to TCISM-ligand on CD3⁺ lymphocytes.

In other embodiments, the cytokine modulator binds selectively to TCISM-receptor on CD68⁺ monocytic cells.

Yet in other embodiments, the clinical condition comprises an autoimmune disease. Within these embodiments, in some cases, the autoimmune disease comprises Rheumatoid Arthritis, Multiple Sclerosis, Crohn's Disease, Psoriasis, Psoriatic Arthritis, Graves Disease, Autoimmune Polyendocrine Syndromes, Hereditary Proteinuria Syndrome, Type I Diabetes, Systemic Lupus Erythematosus, Primary Bilary Cirrhosis, Autoimmune Thyroiditis, Hepatitis, Acquired Immunodeficiency Disease (HIV), Graft versus Host Disease, Allograft Disease, Asthma, Cutaneous T-Cell Lymphoma, HTLV-I-Associated Cutaneous T-Cell Lymphoma, HTLV-II-Associated Lymphoma, Hairy Cell Leukemia, Idiopathic CD4⁺ T-Lymphocytopenia, or Melanoma, or a combination thereof.

Still another aspect of the invention provides a method for treating an autoimmune disease in a subject comprising administering a therapeutically effective amount of an antagonist to a TCISM-ligand or an antagonist to the corresponding TCISM-receptor to the subject in need of such treatment.

Yet another aspects of the invention provides cytokine modulators that can be used to modulate cytokine production by selectively binding to a TCISM-ligand and/or a TCISM-receptor.

### BRIEF DESCRIPTION OF THE DRAWINGS

FIG. 1 is a graph showing that mitogen-stimulated human T-cells can induce monocytic THP-1 Mφ to secrete proinflammatory cytokines through cell-cell contact.

FIG. 2 is a graph showing different activation stimuli induce different T cell “TCISM Ligand” activities.

FIG. 3 is a graph showing human “cytokine cocktails” induce human TCISM-ligand expression in PBMC-derived human T cells.

FIG. 4 is a graph showing cytokine cocktail-activated human PBMC-derived Pan CD3⁺ T cells do not activate human THP-1 cells to produce TNF-α.

FIG. 5 is a graph showing that elevated levels of IL-12 (p70) are produced by freshly obtained human blood monocytes after cell-cell contact with TCISM ligand-positive human CD3⁺ T cells.

FIG. 6 is a graph showing that highly-purified stimulated human Hut-78 T cell membranes are more effective in activating human THP-1 M cells to secrete TNF-α then stimulated Hut-78 whole cells fixed with 1% paraformaldehyde.

FIG. 7 is a graph of a microarray 2D cluster of genes encoding membrane or membrane associated proteins in PHA/PMA stimulated CD3⁺ T Cells, Hut-78, H9, Molt4, Jurkat & Raji Cells.

FIG. 8 is a log-log “Signature” gene graph illustrating expression levels of potential human T-cell TCISM-ligand gene candidates.

FIG. 9 is graphs of FACS analysis of PMA/PHA stimulated Hut-78 T-cells or non-stimulated Hut-78 T-cells measuring different known human T-cell membrane costimulation proteins.

FIG. 10 Quantitative Real-time PCR (qRT-PCR) measurements of Total RNA obtained from PMA/PHA stimulated Hut-78 human T-cells for 6 h.

FIG. 11 is graphs showing that neutralizing anti-human monoclonal antibodies to IFN-γ, CD40L, IFN-γ+CD40L, IL-15, or IL-15 receptor (IL-15R) do not inhibit activated Hut-78 T cell purified membrane-driven THP-1 Mφ cell production of TNF-α or IL-1β.

FIG. 12 is a graph of FACS analysis of hygromycin-resistant Flp-In 293 Cells.

FIG. 13 is graphs showing that CD40L+IFN-γ stimulates TNF-α production, but not IL-1β production, using the flp-in molecular analysis procedure.

FIG. 14 is a graph showing inhibition of T-cell-mediated TNFα production from human Mφ (i.e., adaptive immunity) in some instances, without having any effect on LPS-stimulated TNFα and IL-1β production (i.e., innate immunity).

FIG. 15 is a schematic illustration of one possible mechanism of p.

FIG. 16 is a graph showing assessment of different small molecule TNFα inhibitors in Murine Collagen-Induced Arthritis in mice.

FIG. 17 is a graph showing efficacy of anti-TNFα and IL-1ra treated mice.

FIG. 18 is a slide of joint histopathology of representative mice.

FIG. 19 is Table 1 showing a list TCISM-ligands.

### DETAILED DESCRIPTION OF THE INVENTION

It is believed that one of the mechanisms of activation for T_{c}-induced Mφ activation is via direct cell-cell contact through an immune synapse mechanism. Activated T-cells express numerous known and unknown membrane-bound proteins. The present inventors have discovered that some of these molecules, which are referred herein as T_{c} Cytokine Inducing Surface Molecules (i.e., TCISM or TCISM-ligand), are involved in cell-cell contact signaling cascades leading to proinflammatory cytokine induction by selectively binding to a corresponding TCISM-receptor that is present in Mφ.

A wide variety of clinical conditions are mediated by acute or chronic inflammation including, but not limited to, Rheumatoid Arthritis, Multiple Sclerosis, Crohn's Disease, Psoriasis, Psoriatic Arthritis, Graves Disease, Autoimmune Polyendocrine Syndromes, Hereditary Proteinuria Syndrome, Type I Diabetes, Systemic Lupus Erythematosus, Primary Bilary Cirrhosis, Autoimmune Thyroiditis, Hepatitis, Acquired Immunodeficiency Disease (HIV), Graft versus Host Disease, Allograft Disease, Asthma, Cutaneous T-Cell Lymphoma, HTLV-I-Associated Cutaneous T-Cell Lymphoma, HTLV-II-Associated Lymphoma, Hairy Cell Leukemia, Idiopathic CD4+ T-Lymphocytopenia, and/or Melanoma. The present inventors have also found that these clinical conditions can be treated by modulating cytokine production in monocyte lineage-derived macrophages by administering a cytokine modulator that can selectively bind to a TCISM-ligand of T lymphocytes and/or the corresponding TCISM-receptor of monocyte lineage-derived cells or by inhibiting the transcription of TCISM-ligands, e.g., by iRNA or siRNA.

Some aspects of the invention provide TCISM-ligand and methods for modulating cytokine production in monocyte lineage-derived cells of a subject by administering a cytokine modulator that selectively binds to a T-cell cytokine-inducing surface molecule (TCISM)-ligand of T lymphocytes or the corresponding TCISM-receptor of monocyte lineage-derived cells or by inhibiting the transcription of a TCISM-ligand.

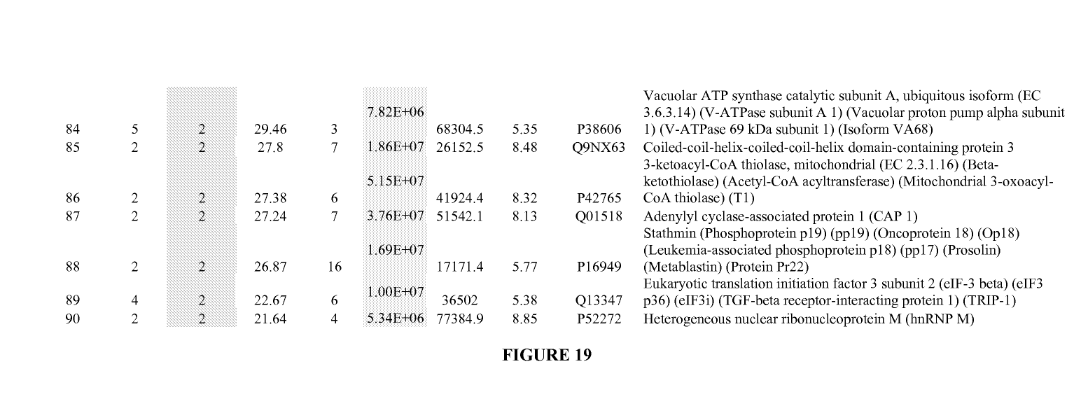

In some embodiments, TCISM-ligand comprises at least one of the TCISM-ligands listed in Table 1 (FIG. 19). The corresponding TCISM-receptor(s) for these TCISM-ligands can be readily determined by one skilled in the art. For example, by the use of neutralizing monoclonal or polyclonal antibodies to inhibit TCISM-ligand to TCISM receptor interactions using the cell-cell contact bioassay. Another method is Subtractive Immunization using stimulated and non-stimulated Hut-78 and H9 subclone T cell membranes to identify monoclonal antibodies that block cell-cell contact. Without being bound by any theory, it is believed that the contact-mediated activation of monocyte-macrophages is a major pathway inducing cytokine production. Accordingly, the modulation of this mechanism, e.g., the blockade of IL-1 and TNF-α production at the triggering level or the inhibition of expression of a TCISM-ligand or TCISM-receptor (e.g., via siRNA), can be used to treat clinical conditions mediated by cytokine production.

Some compositions and methods of the invention are useful in selectively binding a TCISM-receptor that is present in monocyte lineage-derived cells (or by inhibiting expression or transcription of such a receptor), thereby modulating cytokine production in these monocyte lineage-derived cells. Monocyte lineage-derived cells include any cells that when activated by T lymphocytes produce a cytokine. In some embodiments, compositions and methods of the invention modulate proinflammatory cytokine production.

Exemplary monocyte lineage-derived cells that produce a cytokine include, but are not limited to, monocyte lineage-derived macrophages, antigen-presenting cells (APC), dendritic cells, Langerhans cells, and Kuppfler Cells.

Compositions and methods of the invention include molecules that can selectively bind to a TCISM-ligand or TCISM-receptor. In addition, compositions and methods of the invention also include molecules that can modulate translation, transcription, and/or expression of TCISM-ligand or TCISM-receptor. For example, siRNAs can be administered to T lymphocytes to modulate expression of TCISM-ligands. By knowing appropriate TCISM-ligands, one skilled in the art can readily identify appropriate siRNAs that can modulate the expression of TCISM-ligand. For example, siRNA's to the messenger RNA coding for a full-length protein can be designed with commercially available computer software which allows one to determine the sections of mRNA most susceptible to destabilization during transcription.

Controlling adaptive (acquired) immunity at the level of TCISM is advantageous since therapeutic intervention allows for innate (natural) immunity during bacterial skin infections. Accordingly, some aspects of the invention provide compositions and methods for modulating adaptive or acquired immunity while substantially maintaining innate immunity.

Exemplary TCISM-ligand, TCISM-receptor and/or cytokine modulator compounds of the invention include, but are not limited to, compounds having the following formula, analogs and derivatives thereof:

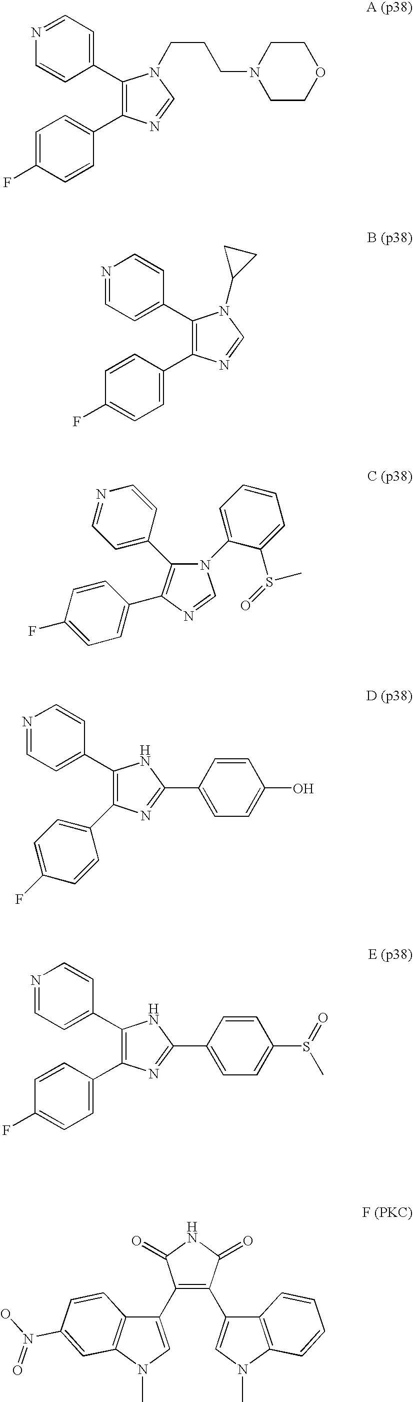

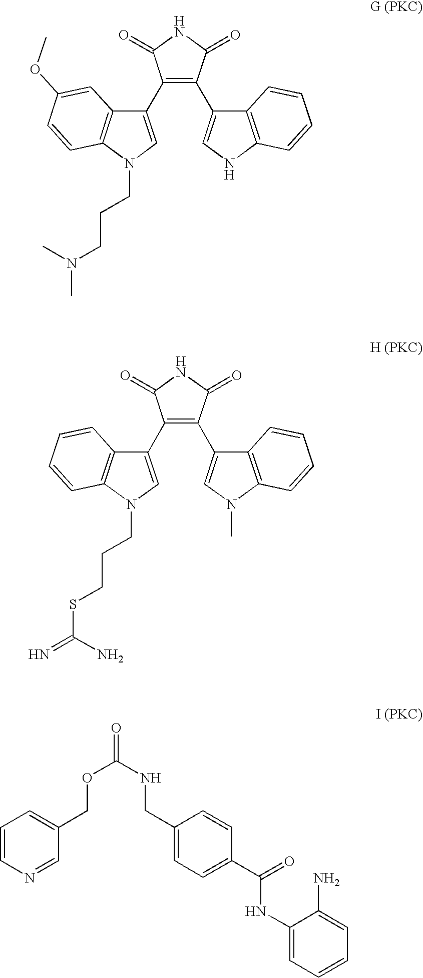

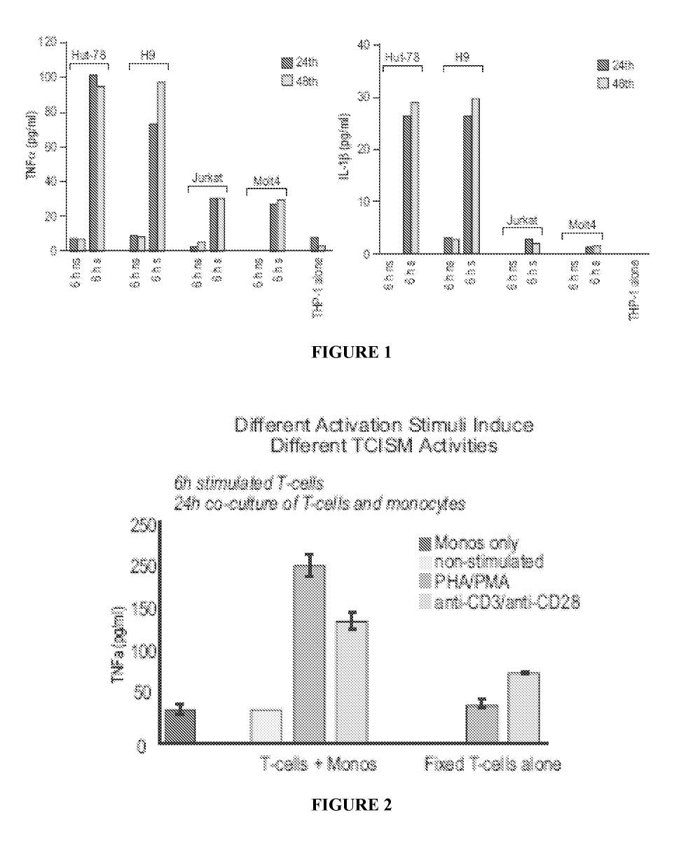

A highly-reproducible and validated cell-cell contact bioassay was established using either primary human T-cells and autologous freshly obtained human blood monocytes, or human T-cell and monocyte cell lines. FIG. 1 is a graph showing mitogen-stimulated human T-cells can induce monocytic THP-1 Mφ to secrete proinflammatory cytokines through cell-cell contact. FIG. 1 is a time course measurement of cytokines at 24 h and 48 h of cell-cell contact. TNF-α (left panel) and IL-1β (right panel) production in THP-1 Mφ were incubated with different PMA/PHA stimulated human T-cell lines. Cells from the monocytic line THP-1 were incubated with highly-purified membrane preparations from stimulated (“s”) or non-stimulated (ns) resting T cells; cytokine production was measured 24h or 48 hours after incubation. A significant increase in TNF-α and IL-1β production was detected from THP-1 Mφ incubated with sHut-78 and sH9 T-cells at both time periods. Means+/−standard deviation with triplicate measurements are provided. As shown in FIG. 1, only a small induction of TNF-α and IL-1β production was observed in PHA/PMA stimulated Molt4, Jurkat and Raji cells, while no detectable production was observed in resting primary human T-cells, unstimulated Hut-78, H9, Molt4, Jurkat or Raji cells. It was also observed that the PMA/PHA-stimulated H9 cells, and to a lesser extent, the Hut-78 cells, induced the greatest THP-1 Mφ secretion of both TNF-α and IL-1β, indicating that these cells are TCISML positive.

PMA/PHA stimulus provided a significant induction of TCISML on the human T cells since elevated levels of TNF-α were produced in culture over the 24 h culture period. See FIG. 2. In FIG. 2, Pan CD3+ T cells were isolated from healthy human donor blood, stimulated for 6 h at 37° C., washed, and fixed with 1% paraformaldehyde (6 h RT). Cells were then rinsed with PBS, and kept overnight at RT. PBMC-derived CD14+ Mφ were then added and incubated at 37° C. 24 h. Cell culture supernatants were centrifuged, filter sterilized and measured for cytokines by ELISA. Means±SEM are shown (N=6 individual donors with triplicate measurements; P<0.05 in comparison to non-stimulated T cells). Comparable data were observed with T cells stimulated in vitro with αCD3/αCD28. Nearly twice as much TNF-α was produced in the T cell and Mφ co-culture system in comparison to the whole T cell control cultures alone.

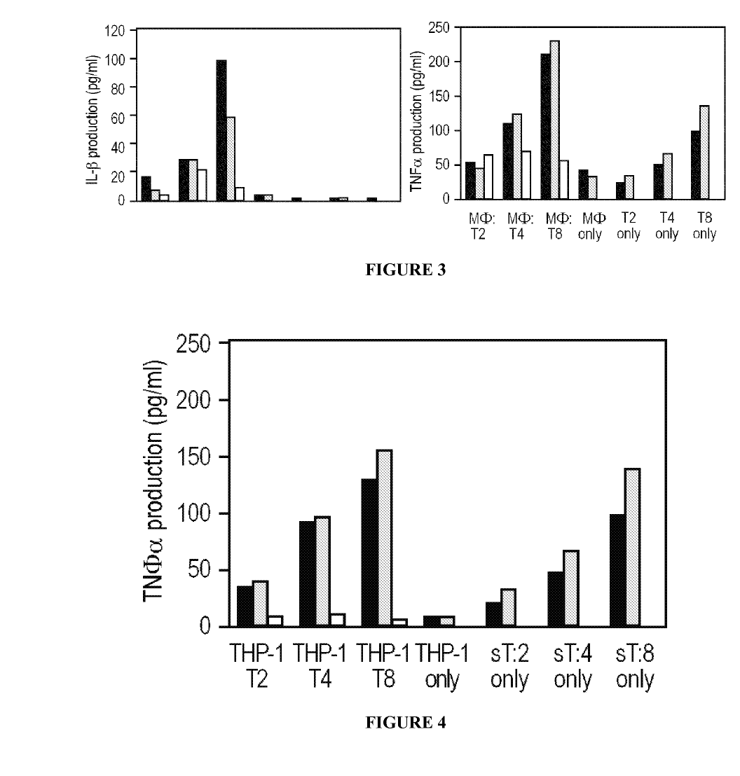

As shown in FIG. 3, the results indicate that cytokine mixture #1 (IL2+IL6+TNF-α) or #2 (IL15+IL6+TNF-α) activated human T-cells, when mixed at a ratio of 1:8 (blood monocyte:T-cells), can activate human monocytes to produce elevated levels (˜100-250 pg/ml) of TNF-α and IL-1β. These cytokine levels were significantly higher than the levels of proinflammatory cytokines released by cytokine-activated, fixed T-cells alone. In FIG. 3, Pan CD3⁺ T cells were isolated from healthy human donors and incubated with different cytokine cocktails (▪IL2+IL6+TNF-α; [symbol] IL-15+IL-6+TNF-α;□ unstimulated T cells) for 8 d at 37° C., washed, and then fixed with fresh 1% paraformaldehyde (6 h RT). Cells were then rinsed with PBS, and kept overnight at RT. Freshly obtained human blood monocytes were subsequently added and incubated with the T-cells (37° C. for 24 h). Cell culture supernatants were collected and measured for cytokines by ELISA. Means±SEM are shown (N=4 individual donors with triplicate measures). Two separate experiments with purified Pan CD3⁺ T-cells from identical donors were conducted to confirm these findings.

Whether human PBMC-derived CD3+ T-cells stimulated with either cytokine mixture #1 or #2 induces the expression of TCISM sufficient to activate human THP-1 cells to produce TNF-α and IL-1β was also tested. As shown in FIG. 4, THP-1 cells combined with cytokine activated T-cells resulted in the production of elevated levels of TNF-α, however, the induced cytokine levels were not significantly higher than those induced by the paraformaldehyde-fixed, cytokine stimulated T-cells alone. In FIG. 4, Pan CD3⁺ T-cells were isolated from healthy human donors and incubated with different cytokine cocktails (▪IL2+IL6+TNF-α; [symbol] IL-15+IL-6+TNF-α; □ unstimulated T cells) for 8 d at 37° C., washed, and then fixed with fresh 4% paraformaldehyde (6 h RT). Cells were subsequently rinsed with PBS, kept overnight at RT, and then added to THP-1 cells (37° C. for 24 h). Cell culture supernatants were collected as above and measured for cytokines by ELISA. Means±SEM are shown.

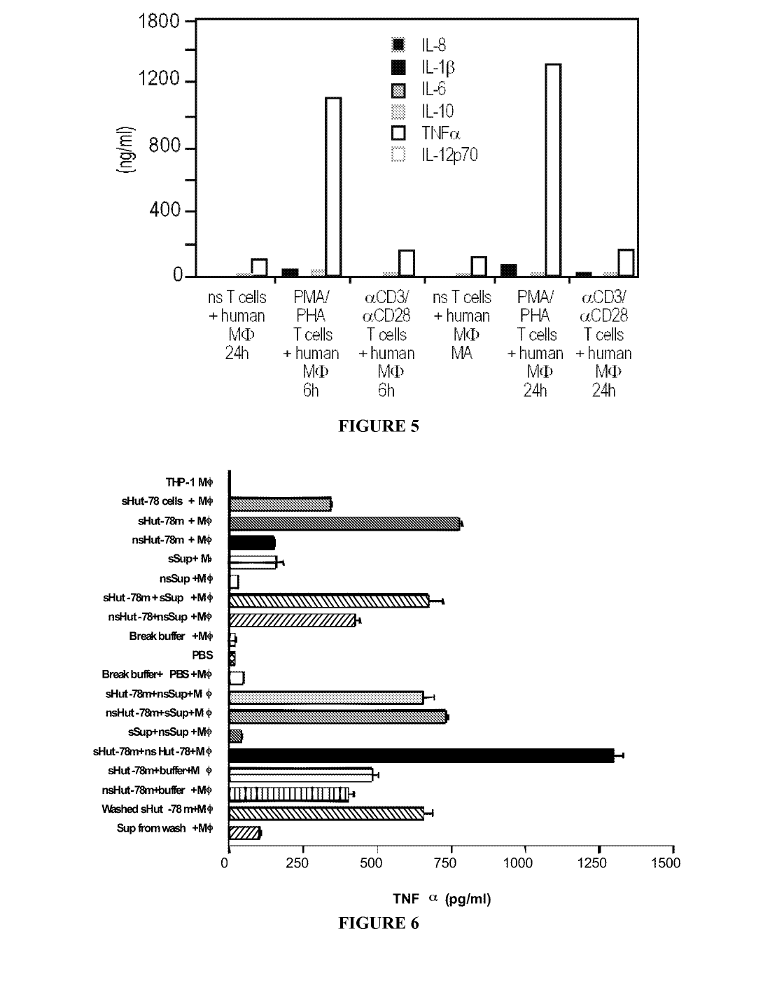

As shown in FIG. 5, Pan CD3+ human T-cells obtained from the peripheral blood of healthy human volunteers were stimulated in vitro with either PMA/PHA or αCD3/αCD28 for 6 h, then fixed with fresh 1% paraformaldehyde overnight at room temperature. Next, freshly obtained human blood monocytes were added to the tissue culture plates and incubated at 37° C. with the fixed T-cells for either 6 h or 24 h of culture. Supernatants were then collected as described above and measured for various Th1 and Th2 cytokines by ELISA. The PMA/PHA stimulated T-cells were potent in activating human blood monocytes to produce IL-12 (p70) at levels ranging between ˜1000-1250 pg/ml, especially after 6 h of cell-cell contact (FIG. 4). Similarly, αCD3/αCD28-stimulated T cells were also effective in activating human blood monocyte IL-12 (p70) release, although to a lesser degree. Finally, both types of stimulated human T-cells were able to activate human blood monocytes to produce IL-1β and TNF-α at these two different time periods.

Ability of PMA/PHA and αCD3/αCD28 stimulated Hut-78 T cells versus PMA/PHA and αCD3/αCD28 stimulated Hut-78 T-cell membranes to activate THP-1 cells to produce TNF-α was compared (see FIG. 6). The PMA/PHA stimulated Hut-78 T-cell data is shown for illustrative purposes. These studies were conducted with the same Hut-78 cell culture lot to reduce variability with the bioassay. Results indicate that stimulated Hut-78 T-cell purified membranes were more effective in inducing in vitro THP-1 cell TNF-α production in comparison to the stimulated Hut-78 fixed with 1% paraformaldehyde. Mix and match “add back” experiments were also conducted to demonstrate that TCISM was predominantly present in the purified membrane fractions and not the Parbomb cell supernatant (e.g., cytosolic) fractions during the low and high centrifugation steps (see FIG. 6). In FIG. 6, membranes were prepared as described above and combined with THP-1 cells in the cell-cell contact bioassay. After 24 h culture, supernatants were removed, centrifuged, filter-sterilized, and measured for cytokines by ELISA. Means+/−SD are shown.

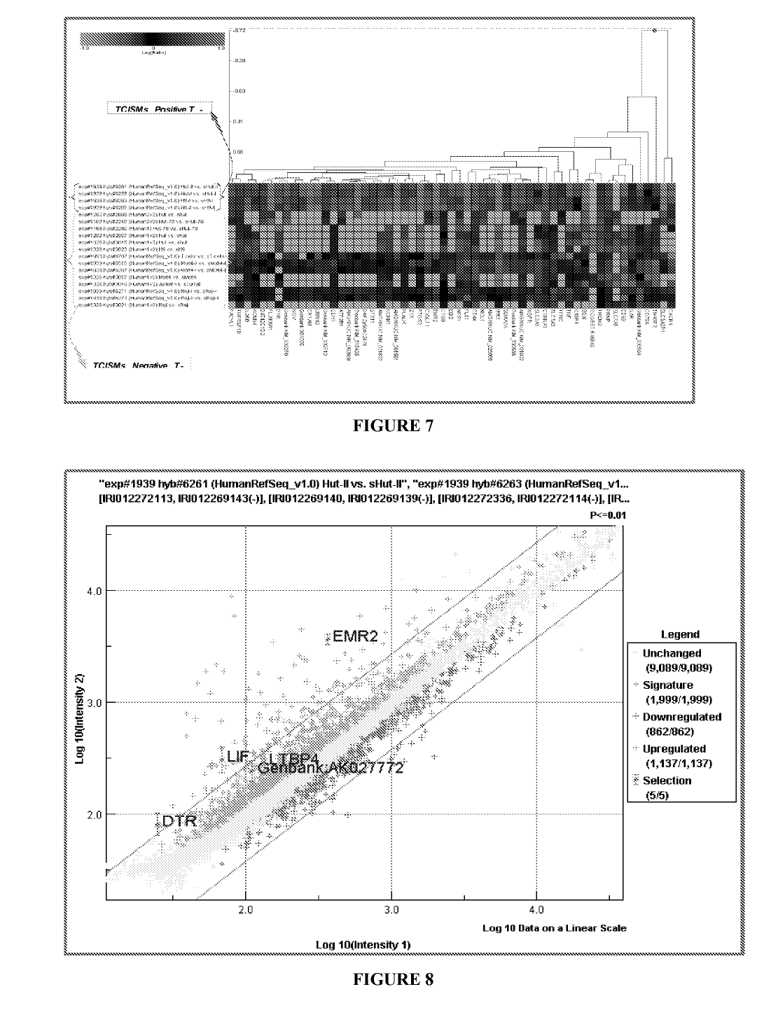

A characteristic array profiling “heat map” is shown in FIG. 7, which is a microarray 2-dimensional (2D) cluster of genes encoding membrane or membrane associated proteins in PHA/PMA stimulated CD3⁺ T Cells, Hut-78, H9, Molt4, Jurkat & Raji Cells. At least four experiments with fold change >2 & P value <0.01 were conducted. In FIG. 7, red represents gene expression levels >2.0 (i.e.; above array profiling background levels), and green represents gene expression levels <2.0 (i.e.; below array profiling background levels). During the computational assessment stage of data review, only those genes that were human T-cell membrane associated and which were up-regulated in the TCISM (+) T-cell lines and down-regulated in the TCISM (−) T-cell lines were considered. Approximately 10,000 out of 50,000 genes resulting from microarray experiments were examined, where the focus centered on T-cell genes which were up regulated in Hut-78 and H9 cells but not up-regulated in human Molt-4 and Jurkat T-cells.

From this curated list of about 100 potential human T-cell TCISMs candidates, log/log intensity plots were generated which were graphed on a linear scale to identify TCISM candidates. These plots (see, for example, FIG. 8), describe “unchanged”, “signature”, “down-regulated”, and “up-regulated” T-cell gene products, as well as the relative expression levels of the gene products compared to overall array profiling results. Five candidate human T-cell TCISM genes were identified: Diphtheria Toxin Receptor, or Heparin-Binding EGF (DTR, EGF module-containing Mucin-like hormone receptor 2 (EMR2), Adamlysin-17 (ADAM or A Disintegrin and Metalloprotease) TNFα-converting enzyme (TACE, TNF receptor Superfamily, member 9 (TNFRSF9 or LIGHT), and, for cell-cell contact positive control purposes driven by review of the published scientific literature, TNFRSF5, or CD40 Ligand (CD40L).

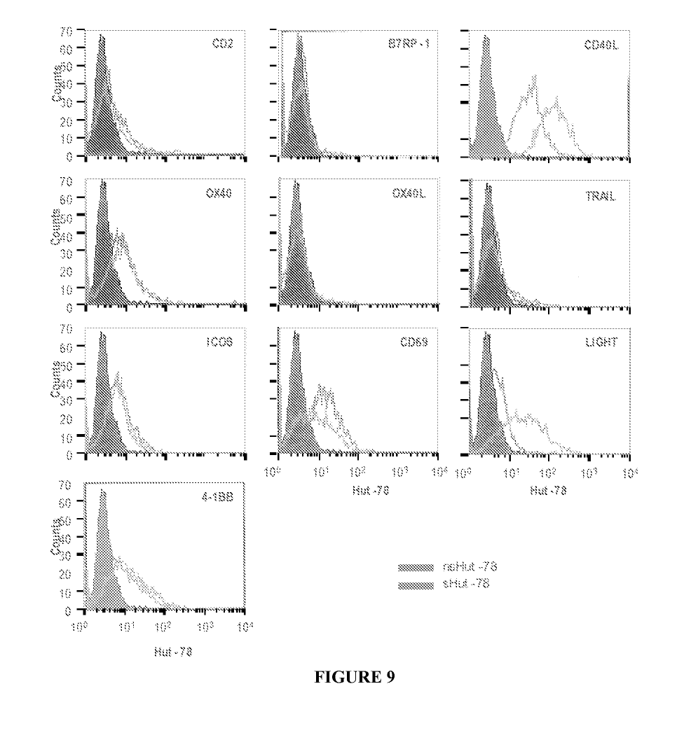

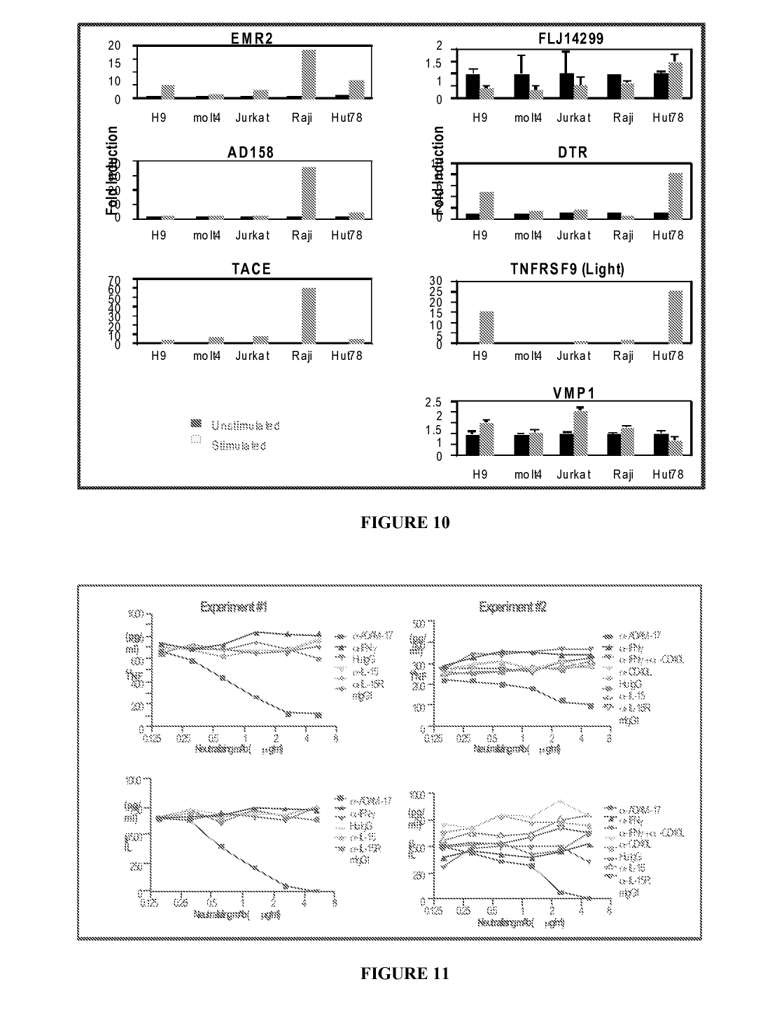

Real-time qRT-PCR was performed in order to confirm TCISM candidate gene expression in PMA/PHA-stimulated Hut-78 and H9 subclone T-cells (see FIG. 9). The TCISM candidates DTR and LIGHT followed preset criteria in that both genes were highly expressed in stimulated Hut-78 and H9 T cells, but were not up-regulated in the Molt-4 and Jurkat T cells or the Raji B-cells. However, both TACE and EMR2 were highly up-regulated in the TCISM (−) human Raji B-cell line. FACS was also used to validate the presence of these molecules on activated H9 cells (see FIG. 10).

About 50 different commercially available neutralizing monoclonal antibodies to known human T-cell surface proteins were tested in the cell-cell contact bioassay (including mAb's directed to ALCAM, CD6, β₂-integrins, CD69, CD23, CD40-CD40L and LAG-3). It was found that they do not appreciably inhibit more than about 30% of the proinflammatory cytokine production in this system. One of the more effective polyclonal antibody preparations observed was anti-ADAM-17 (TACE). These experiments were conducted with available polyclonal antibodies. Use of highly-specific anti-CD40L mAb's in bioassay did not significantly block TNF-α or IL-1β cytokine production (see FIG. 11). In FIG. 11, Hut-78 cells were stimulated with PMA/PHA for 6 h, at which time purified membranes were prepared as described earlier. Hut-78 membranes plus the concentration of anti-human mAb's shown were added to co-culture wells and allowed to incubate at 37° C. for 2 h. Recently passed resident THP-1 cells were then added to co-culture wells, and kept at 37° C. for 24 h. Supernatants were collected for TNF-α and IL-1β ELISA. FIG. 11 shows two separate experiments with triplicate cytokine measurements at each mAb concentration.

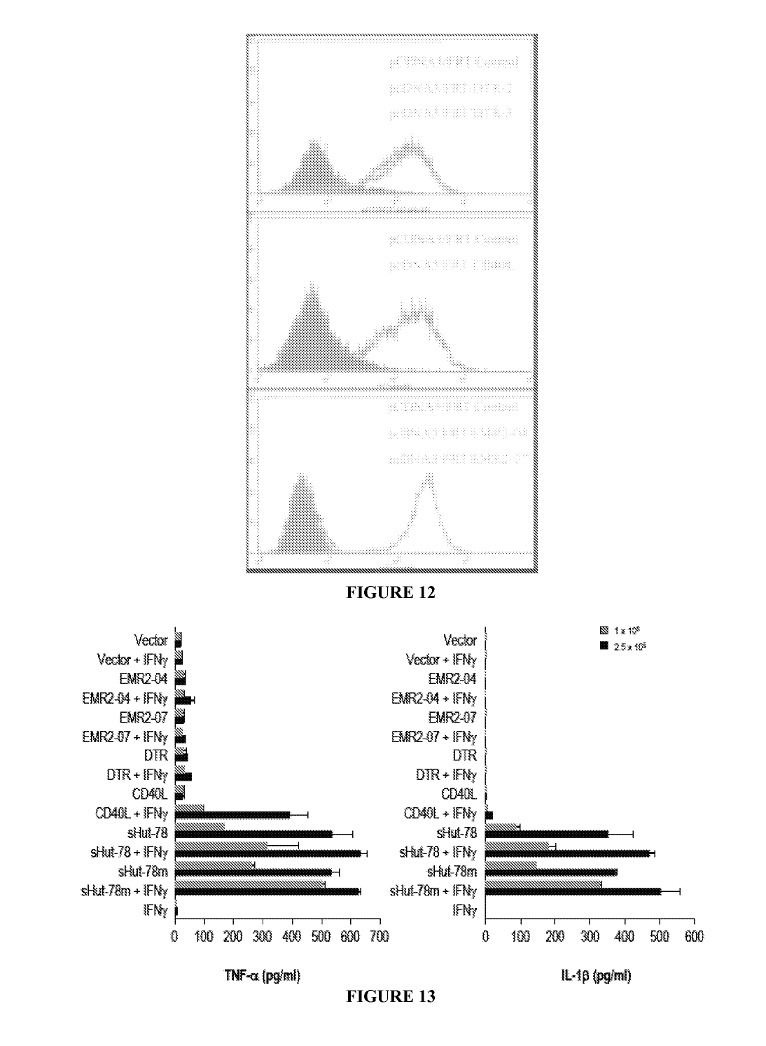

FACS analysis showed that transfected 293 cells (FIG. 12) consistently expressed elevated levels of the TCISML gene product on their cell surface. In FIG. 12, pcDNA5/FRT/DTR, EMR2-07 (isoform containing EGF-like domain 1, 2 & 5), or CD40L were co-transfected with p0G44 into Flp-In 293 cells and Jurkat cells, respectively, and colonies were selected in 500 mg/ml Hygromycin. Cells were detached with cell disassociation buffer and analyzed by FACS. (Anti CD97 was cross-reacted to EMR2-07 and was used for detecting EMR2-07 expression.) Expression was stable after several cell-culture passages, indicating their suitability for use in the cell-cell contact assay. Initial experiments were conducted using the transfected 293 cells in the cell-cell contact assay (see FIG. 13). The DTR and CD40L constructs are potent in augmenting sHut-78m driven THP-1 induction of TNF-α/IL-1β (FIG. 13). The addition of exogenous IFN-γ to the assay resulted in enhanced levels of CD40L-induced Mφ activation and subsequent TNF-α release (FIG. 13 left panel), but not IL-1β release (FIG. 13 right panel). These effects with the CD40L transfected 293 or Jurkat cells were highly reproducible.

Human T-cell TCISMs were identified in both healthy human T-cells and T-cells obtained from patients with active Ps and PsA. As can be seen, the in vitro cell-cell contact bioassay is a highly reproducible human cytokine “readout system” to identify immunological synapse mechanisms between human T-cells and human Mφ in co-culture. It was observed that the PMA/PHA-stimulated H9 cells and the Hut-78 cells induced THP-1 secretion of both TNF-α and IL-1β (indicating that these cells are TCISM positive), while PHA/PMA stimulated Molt4, Jurkat, Raji cells and resting primary human T-cells, unstimulated Hut-78, H9, Molt4, Jurkat or Raji cells are TCISM negative (FIG. 1).

TCISM candidates were determined using the following criteria: (A) membrane-associated; (B) up-regulated by more than 2-fold in stimulated Hut-78 and H9 cells, but not in Molt4, Jurkat and Raji cells; and (C) high expression levels must be confirmed by qRT-PCR and FACS.

Primary T cells stimulated in vitro with αCD3/αCD28 or human cytokine cocktails also augment proinflammatory cytokine (PIC) activity in the assay. PMA/PHA or αCD3/αCD28 stimulated T cells augmented Mφ activation to produce IL-12. IL-12 has been detected in psoriasis lesions. It is believed that IL-12 mainly stimulates IFN-γ production in naïve Th cells and would play a role in the expansion and stabilization of the Th1 response.

Microarray analysis was conducted from PBMC-derived T-cells from normal healthy donors versus psoriasis patients to identify the human T-cell TCISML molecule and its signaling pathways through human Mφ TCISMR that lead to inflammation and cutaneous skin diseases. Microarray analysis using human T cells from healthy donors, PsA, and CPPs patients have identified TCISM molecules, including Diphtheria toxin receptor (DTR; HB-EGF) and mucin-like Epidermal Growth Factor family members (EMR4; CD97). Up-regulation of numerous TNF family members including 4-1BB (TNFSF14), OX-40, LIGHT (TNFSF9) and CD40L (CD154; TNFSF5) was also observed.

About 50 different commercially available neutralizing monoclonal antibodies were used to validate the human TCISM candidates by cell-cell contact assay. In many cases, it was found that these reagents inhibit no more than 30% of the proinflammatory cytokine production over a 24-96 hour time period in this system (see FIG. 11). A “Flip-in” transfection system method was used to identify five initial candidate TCISML gene candidates from the microarray experiments, including: EMR2, DTR, 4-1BB, LIGHT, and CD40L. Full-length cDNA's of TCISM candidate genes transfected into TCISM negative 293 and Jurkat cells, characterized in the cell-cell bioassay, showed that DTR and CD40L were potent in augmenting T cell-driven Mφ TNF-α/IL-1β. The addition of exogenous IFN-γ to the assay resulted in enhanced levels of CD40L-induced Mφ activation and subsequent TNF-α release, but not IL-1β release (FIGS. 12a-b and 13).

Some of the identified human Mφ TCISMRs (TCISM-receptors) that leads to inflammation and cutaneous skin diseases include DTR, CD97, 4-1BB, OX-40, LIGHT and CD40L. Full-length cDNA's of TCISM candidate genes transfected into TCISM negative 293 and Jurkat cells, characterized in the cell-cell bioassay, showed that DTR and CD40L are potent in augmenting T cell-driven Mφ production of TNFα/IL-1β.

### Psoriasis

One particular aspect of the invention provides compositions and methods for treating psoriasis. Psoriasis (Ps) is a chronic skin disorder that affects approximately 2% of the US population. Without being bound by any theory, and as schematically illustrated in FIG. 15, it is believed that the pathophysiology of Ps involves epidermal proliferation and differentiation, angiogenesis and hyperproliferation of keratinocytes and infiltration of activated T-cells (T_{c}), macrophages (Mφ), dendritic cells (DC), Langerhans cells (LC) and neutrophils (PMN) into lesional skin. Proinflammatory cytokines (PIC), including Tumor Necrosis Factor-α (TNFα), Interleukin-1β (IL-1β), and Interleukin-32 (IL-32), produced in the active lesion, are believed to induce and maintain chronic skin inflammation in diseases such as psoriatic arthritis (PsA) and Ps. Up-regulation of cytokines by activating Mφ is believed to be responsible for the pathogenesis of these disorders. It has been suggested that T-cells play a key role in Mφ activation; however, T_{c} cytokines, such as Interleukins-4, 10, and 13 (IL-4, IL-10 and IL-13), have been shown to either play an anti-inflammatory role or only weakly induce TNFα/IL-1β up-regulation. It is believed that the mechanism of activation for T_{c}-induced Mφ activation is via direct cell-cell contact through an immune synapse mechanism in the skin.

An immunological synapse (IS) is formed at the interface between antigen-presenting cells (APCs) and T-cells, and is believed to be the structure responsible for antigen recognition and T-cell activation. The IS was initially found between T-cells and B-cells, or between T-cells and MHC-containing planar bilayers. It is believed to be formed by the accumulation of the T-cell receptor-major histocompatibility complex (TCR-MHC) in the IS central region, termed the central supramolecular activation cluster (c-SMAC), and the accumulation of leukocyte function-associated antigen 1 (LFA-1)-intercellular adhesion molecule-1 (ICAM-1) in external IS regions, termed the peripheral (p-) SMAC (pSMAC). The mature IS has been shown to contain a pSMAC that is enriched with LFA-1, talin, VLA-4, ADAP and transferring receptor. The pSMAC surrounds the cSMAC, which is enriched with the TCR, CD4 or CD8 co-receptors, CD28 co-stimulatory molecules, CD2, PKCθ, etc.

Ps skin is characterized by the hyperproliferation of keratinocytes, resulting in an exaggerated pattern of ridges and pegs. Keratinocytes, DC, and Mφ in skin have all been shown to produce TNFα, IL-1β, and IL-32. While the IS controls psoriatic autoantigen-specific cutaneous lymphocyte antigen (CLA)-positive T-cell activation, the key molecular components include the TCR and, surrounding it, a ring of adhesion molecules, such as LFA-1, which can bind to ICAM-1 expressed by the adjacent cell, e.g., a keratinocyte or APC. The IS is therefore a logical target for therapeutic approaches. Alefacept, for example, is a recombinant fusion protein that binds to CD2 on memory-effector T-cells, inhibiting their activation and reducing the number of these cells. Efalizumab is a humanized monoclonal antibody (Mab) against CD11a molecule. CD11a and CD18 comprise subunits of LFA-1.

The present inventors have shown that there exist TCISMs on the surface of human T-cells which mediate skin inflammation by driving Mφ activation to produce proinflammatory cytokines Controlling adaptive (acquired) immunity at the level of TCISM is advantageous since therapeutic intervention allows for innate immunity during bacterial skin infections.

Psoriasis is a hereditary disorder of the skin with several clinical expressions. The most frequent type is psoriasis vulgaris (or Plaque Psoriasis [Ps]), which occurs as chronic, recurring, scaling papules and plaques in characteristic sites on the body. Current therapies for psoriasis are not satisfactory. Ps is characterized by the infiltration of the skin by activated T-cells and an abnormal proliferation of keratinocytes. As a result of overproduction by T-cells, keratinocytes, DC, and LC, it has been reported that the concentrations of TNFα are higher in Ps lesions than in uninvolved skin (in both patients with Ps and normal persons).

### Autoimmune Diseases

Autoimmune diseases in humans, such as Ps and Psoriatic Arthritis (PsA), are chronic syndromes characterized by typical, often relapsing clinical symptoms combined with diagnostic results of adaptive humoral (autoantibodies) or cellular (autoreactive T-cells) responses directed against autoantigen-expressing tissues. Important human autoimmune diseases often are co-morbid with, or are triggered by, viral or bacterial infections and are associated with certain MHC alleles. The innate immune system encompasses a collection of host defenses that range from non-specific barrier function of epithelia to the highly selective recognition of pathogens through the use of germline-encoded receptors. A common feature of these diverse elements is a rapid and blunt response to infection or tissue destruction. On the other hand, the adaptive immune system uses somatically rearranged antigen receptor genes to create receptors for virtually any antigen. The adaptive immune response is slower but more flexible and is able to combat infections that have evolved to evade innate responses.

The innate immune system responds by recognition of conserved motifs in pathogens as well as a number of other indictors of cell stress or death. The cellular components of the innate immune system includes DC, monocytes, Mφ, granulocytes and natural killer T-cells (NKT), as well as the skin, pulmonary, and gut epithelial cells that form the interface between an organism and its environment. The non-cellular elements of the innate system are very diverse, and range from the simple barrier function of the stratum corneum to complex pathways such as the complement cascade. These elements prevent entry of pathogens through physical blockade, or, once cells are invaded, allow them to destroy pathogens directly or via phagocytic cells. The innate immune system has also evolved to recognize molecular patterns common to many classes of pathogens. These are termed pathogen-associated molecular patterns (PAMPs). PAMP recognition is through using a group of germ line-coded, evolutionary conserved pathogen-recognition receptors (PRR). The Toll-like receptors (TLR) are a very important group of pathogen receptors, and they are expressed on both innate immune cells and on cells in various tissues, including endothelial cells, epithelial cells, and fibroblasts. Ten TLR family members specific for various microbial molecules have been identified in humans. Binding of TLR to their microbial ligands leads to activation of phagocytes, as well as to the release of proinflammatory cytokines and anti-microbial peptides. These molecules are believed to also activate DC to initiate adaptive immune responses.

T-cells are important in immune response and can be divided into a number of distinctive subsets based on their migration patterns and functional abilities. Naïve T cells recirculate primarily between the blood and lymph nodes, a pattern aided by their expression of the homing receptors L-selectin and CCR7. Naïve T-cells are maintained in a pluripotent state and have a relatively quiescent effector program as they recirculate from blood through lymphoid organs, surveying DC for activating MHC-peptide complexes. Through complex mechanisms that integrate signals from activated DC and from the cytokine milieu, naïve T-cells are driven through rapid rounds of division that are linked intimately with the ability to secrete effector cytokines necessary to confront distinct groups of pathogens. CD4⁺ T helper cells can be functionally divided into Th1 (interferon [IFN]γ-secreting) and Th2 (interleukin [IL]-4-secreting) subsets, as well as recently identified additional Th subsets which include Tr1 (IL-10-secreting), Th3 (transforming growth factor [TGF]β-producing), ThFH (follicular helper cells), peripherally-induced T regulatory (Treg; FoxP3-positive) and Th17 (IL-17A-producing) cells. The discovery of additional subsets will undoubtedly fuel interest in identification of underlying regulatory transcription factors that are likely to be implicated in mechanisms that modify the signature cytokine genes involved in effector function.

An immunological synapse (IS) is formed at the interface between antigen-presenting cells and T-cells, and is believed to be the structure responsible for antigen recognition and T-cell activation. The IS was originally found between T-cells and B-cells, or between T-cells and MHC-containing planar bilayers. It is formed by the accumulation of T-cell receptor-major histocompatibility complex (TCR-MHC) in the central IS region, termed the central supramolecular activation cluster (c-SMAC), and the accumulation of leukocyte function-associated antigen 1 (LFA-1) intercellular adhesion molecule-1 (ICAM-1) in external regions, called the peripheral (p-) SMAC (pSMAC). The mature synapse contains a pSMAC that is enriched with LFA-1, talin, VLA-4, ADAP and transferring receptor. The pSMAC surrounds the cSMAC, which is enriched with the TCR, CD4 or CD8 co-receptors, CD28 co-stimulatory molecules, CD2, PKCO, etc.

Ligands expressed on the surface of the APC are believed to recruit specific receptors to the IS contact site. The recruitment of co-stimulatory molecules CD28 and cytotoxic T lymphocyte antigen 4 (CTLA4) to the synapse is differentially promoted by the expression of their ligands, B7-1 and B7-2, on the APC. Although CD28 and CTLA4 bind either of these ligands, when expressed on the APC, B7-2 recruited CD28 and B7-1 recruited CTLA4 to the synapse. Stability of the ligand in the contact site on the APC is also important, as the recruitment of CD28, CTLA-4 and protein kinase C-θ require the presence of the cytoplasmic domain of B7-1.

Psoriatic skin is characterized by the hyperproliferation of keratinocytes, resulting in an exaggerated pattern of ridges and pegs. Keratinocytes, DC, and Mφ in skin can all produce TNFα. While the IS controls psoriatic autoantigen-specific cutaneous lymphocyte antigen (CLA)-positive T-cell activation, some of the key molecular components include the TCR and, surrounding it, a ring of adhesion molecules, such as LFA-1, which can bind to ICAM-1 expressed by the adjacent cell, e.g., a keratinocyte or APC. The LFA-1 component of the synapse is believed to be important in psoriasis, as a therapeutic agent (anti-LFA-1 antibody; efalizumab) blocking this adhesive interaction has been approved by the US Food and Drug Administration (FDA) for the treatment of psoriasis. Additional contributory molecules, including, other adhesion molecules and co-stimulatory molecules, also influence T-cell responsiveness, e.g., the cell surface molecular pairs CD2:LFA-3 and CD28:CD80/CD86.

### Rheumatoid Arthritis

Rheumatoid Arthritis (RA) is an inflammatory disease also related to IS signaling. One of the potential approaches for the treatment of RA involves the inhibition of molecules present at the IS between T-cells and antigen-presenting cells. It is believed that the mechanism of cytokine up-regulation is contact-dependent. There are multiple candidate proteins on the cell surface that can mediate these functions.

It has been shown that T-cells activated through the T-cell receptor complex induce monocyte IL-10 synthesis. This is partially dependent on endogenous TNFα and IL-1 levels, and T-cell membrane TNFα has been shown to be an important contact-mediated signal. However, IL-10 synthesis still occurs when TNFα and IL-1 are neutralized, thus indicating that there are TNF/IL-1-independent signals required for IL-10 synthesis.

Of particular interest are members of the TNF/TNF-R family, which include CD40, CD27, CD30, OX-40, and LTβ. The ligands of these TNF-R molecules are believed to be upregulated upon T cell activation and, in addition, CD40L, 4-1BB, CD27L, CD30 are believed to be released as soluble mediators after activation. The interaction between CD40L and CD40 has been observed to be of importance for inducing both IL-1 and IL-12 synthesis following T-cell interaction with monocytes, and more recently, to mediate IL-10 production by human microglial cells upon interaction with anti-CD3-stimulated T cells.

### T-Cell Immunoglobulin Mucin Proteins

The T-cell immunoglobulin mucin (TIM) proteins are type I membrane glycoproteins expressed on T-cells that contain common structural motifs. The TIM gene family is located on chromosome 11 in mice and 5q33 in humans. Genomic analysis has identified eight family members in mice (TIM-1 to TIM-8) and three in humans (TIM-1, TIM-3 and TIM-4). All members share a characteristic structure containing IgV, mucin, transmembrane, and cytoplasmic domains. This gene family plays a role in the regulation of immune responses.

TIM-1, previously identified as the hepatitis A virus receptor, co-stimulates T-cell expansion and cytokine production. TIM-1 is expressed on all activated T cells and, upon CD4⁺ T-cell polarization, at a higher level on Th2 than on Th1 cells. An agonistic monoclonal anti-TIM-1 antibody (3B3) was shown to costimulate T-cells in vitro when cultured with either peptide and APCs or cross linking antibodies against CD3 and CD28. When administered in vivo during an immune response, anti-TIM-1 antibody augmented T-cell proliferation in vitro, even in the absence of antigenic re-stimulation. It also increased the production of both Th1 and Th2 prototypic cytokines compared with control treatments. In addition, anti-TIM-1 antibody abrogated the induction of high-dose tolerance and could also restore AHR when mice were immunized and challenged with antigen intra-nasally. TIM-1 is therefore surmised to act as a co-stimulatory molecule for all T-cells, with possibly stronger effects on Th2 than Th1 cells.

It has been demonstrated that TIM-3 is preferentially expressed on in vitro polarized human CD4⁺ Th1 cells as compared with Th2 cells. Thus, TIM-3 expression can be used to identify human Th1 cells. In addition, TIM-3 is believed to contribute to regulation of Th1 cells in vivo. For example, administration of TIM-3-specific antibody to mice in an experimental autoimmune encephalitis (EAE) model resulted in the acceleration of a Th1-driven progression of EAE. Additionally, anti-TIM-3 antibodies induced Mφ activation and clonal T-cell expansion, for which a cognate interaction between Mφ and T-cell was required. These data appear to show a role for TIM-3 in negatively regulating the activation of Mφ by T-cells. TIM-3/TIM-3 ligand interactions also play a role in tolerance. Treatment of mice with both full-length and soluble TIM-31 g fusion proteins abrogates tolerance induced using high-dose aqueous antigen. Similarly, TIM-3-deficient mice cannot be tolerized. Indeed, both Ig fusion protein-treated mice and TIM-3-deficient mice exhibit increased T-cell proliferation and production of IL-2 after administration of high-dose aqueous antigen relative to controls.

TIM-4 is believed to be a natural ligand for TIM-1. Unlike the other TIM molecules, TIM-4 does not appeared to be expressed in T cells but is instead appears to be expressed in APCs, particularly in mature lymphoid DCs. A positively regulating TIM-like family molecule which mediates Th1 T-cell-driven Mφ activation has not been discovered to date. The present inventors have shown that neither TIM-1 nor TIM-3 is up regulated in PMA/ionomycin-activated H9 or primary human CD3⁺ T cells.

Proteomic Approaches to Identify Cytoplasmic, Membrane, and Nuclear proteins Involved in the Immunological Synapse

Proteomics technologies can be used to characterize biomarkers and biosignatures of disease and to reveal information regarding functional subproteomes and networks. Although many proteomics applications provide general information about subsystems that change in response to disease, insult or drugs, proteomics can also be used to identify previously uncharacterized proteins involved in biochemical responses such as MAPK signaling, chemotaxis, melanoma oncogenesis and metastasis, and MHC Class II-induced cell death, etc. The combination of two-dimensional gel electrophoresis (2DGE) and mass spectrometry is one of the analytical techniques used for proteomics applications. The quantitative capability of 2DGE, coupled with the direct and unbiased identification of proteins via tandem mass spectrometry and advanced database searching algorithms, provides an excellent technical platform with which to address the profiling of protein expression changes in cell system, plasma, skin, etc. A complementary discovery platform is multi-dimensional chromatographic protein and peptide separations followed by tandem mass spectrometry and database searching for protein identification. Selected reaction monitoring is subsequently used to quantify relevant molecules.

### Host Defense and Inflammatory Disease

The proinflammatory, pleotrophic cytokines TNFα and IL-1β play key roles in host defense and inflammatory disease processes. TNFα and IL-1β overexpression has been found in Ps disease target tissue as well as the circulation of patients with inflammatory skin diseases. One of the major functions of T-cells and monocyte-M4 is to release various cytokines, including IL-1β and/or TNFα. These molecules, in turn, participate in the induction and release of downstream moieties such as IL-32 (produced by keratinocytes and Mφ), eventually leading to keratinocyte hyperproliferation and the development of Ps. Without being bound by any theory, it is believed that blocking the production of these cytokines at a more distal level (e.g., at the level of T-cell driven monocyte-Mφ activation, perhaps at different time periods during the adaptive response and/or IS formation), can lead to new, molecular drug discovery targets designed to specifically inhibit these cytokines, resulting in safer therapeutics with less adverse events for Ps patients.

Additional objects, advantages, and novel features of this invention will become apparent to those skilled in the art upon examination of the following examples thereof, which are not intended to be limiting.

### Examples

### Culture of Cell Lines

The human cell lines were cultured in a standard medium consisting of RPMI 1640 (Biochrom, Berlin, Germany) supplemented with 10% (v/v) FCS serum, 2 mM L-glutamine, 100 units/ml penicillin and 100 units/ml streptomycin. Hut78, H9, Molt4, Jurkat, Raji and THP-1 cell lines were obtained from the ATCC.

### Cell Isolation

Peripheral blood mononuclear cells (PBMCs). PBMCs were isolated by Ficoll density gradient centrifugation. The viability of obtained PBMCs was >95%, as determined by trypan blue staining The viable cells were quantified in a Neubauer chamber (Zeiss, Oberkochen, Germany) and stored in liquid nitrogen.

### CD3+ T Cells

Thawed PBMCs were centrifuged again at 1,500 rpm for 5 minutes. Supernatant was discarded and pellets were resuspended in MACS buffer. The cells were disrupted into a single cell suspension at a concentration of 0.8 ml of buffer per 10⁸ cells. About 0.2 ml of Hapten-Antibody Cocktail per 10⁸ cells was added. The resulting mixture was mixed well and incubated for 20 minutes on ice. Cells were washed by adding 20× the labelling volume, centrifuging and supernatant removal. Cell pellets were resuspended in 0.8 ml of buffer per 10⁸ cells. About 0.2 ml of MACS Anti-Hapten MicroBeads per 10⁸ cells was added to label the cell magnetically. The mixture was incubated for 15 minutes on ice, washed with 20× of the volume (frozen cells from Leukophoresis packs were passed through a 45 μm mesh filter to remove clustered dead cells), centrifuged and supernatant discarded. Cells were resuspended in 1 ml of MACS buffer per 10⁸ cells and the LS+ column placed in the magnetic field of an appropriate MACS separator. The column was prepared by washing with 3 ml of buffer. The cell suspension was applied to the column and the unlabeled cells were passed through. The effluent was collected as a negative fraction, representing the enriched T cell fraction. The column was rinsed with 4×3 ml of buffer and effluent collected. Following cell washing, the cell pellet was resuspended in 50 ml of tissue culture medium at a concentration of 10⁶ cells/ml. T cell purity (>95%) was determined by CD3-FITC labelling and FACS analysis.

### Monocytes

Method was the same as CD3+ T cell separation.

### Immunophenotyping of the Cells

Harvested cells were washed in FACS medium [phosphate buffered saline (PBS) containing 1% bovine serum albumin (BSA)] and stained at 4° C. for 20 min by antibodies directly conjugated with Fluorescein isothiocyanate (FITC) or phycoerythrin (PE). Thereafter cells were washed three times with PBS and analyzed by FACScan (Becton Dickinson, Heidelberg, Germany) using the CellQuest software (Becton Dickinson). Antibodies were the following: PE-labeled anti-mouse IgG, anti-human CD40L and CD137.

### Cell-Cell Contact Assay

Primary T cells or H9 cells were washed with cold PBS and cell pellets were resuspended in freshly made 1% paraformaldehyde at 5×10⁶ cells/ml. Cells were fixed on ice for 2 hours, and then washed with 20× volume of cold PBS three times. Following the third wash, the cells were kept in PBS at 4° C. overnight to allow diffusion of paraformaldehyde. Cells were centrifuged and washed one more time. The fixed T cells were resuspended in medium at 1×10⁷ cells/ml and dispensed into a 96 well U bottom plate at about 1×10⁶/ml THP-1 cells 100 μl per well. Primary T cells or H9 cells (100 μl per well) were added at 8×, 4×, and 2×10⁶ cells/ml. The plates were incubated at 37° C., in humidified 5% CO₂ for 48 hours. Plates were centrifuged at 1,500 rpm for 5 minutes and supernatant (120 μl) transferred into a fresh plate. The supernatant was stored at −20° C. until used.

### Hut-78 Plasma Membrane Preparation

About 5×10⁸ stimulated or unstimulated Hut-78 cells were suspended in 10 ml of hypertonic buffer containing protease inhibitors (50 mM Tris-Cl (pH 7.4), 25 mM KCl, 5 mM MgCl₂, 200 uM PMSF, 1× complete protease inhibitor) and homogenized using a dounce homogenizer by 20 strokes on ice. The nucleus and unbroken cell fraction were discarded by centrifugation at 4000×g at 4° C. for 15 min and the supernatant was ultracentrifugated at 28K (100,000×g) using a SW40 rotor at 4° C. for 45 min. The membrane pellet was resuspended in 9 ml of PBS with a 22G syringe needle and was then added to 1 ml of 200 mM CHAPS. The homogenate was incubated on ice for 1 hr. Approximately 1 ml aliquots of suspended plasma membrane at 5×10⁷ cell equivalent/ml was stored at −80° C.

### RNA Sample Preparation and Hybridization

RNA was extracted and purified using the RNeasy MinElute kit (Qiagen) and Qiagen Mini RNeasy kit according to the manufacturer's protocol. cDNA synthesis was carried out as described in the Expression Analysis Technical Manual (Affymetrix, two-cycle protocol) using 100 ng of total RNA for each sample. The cRNA reactions were carried out using the BioArray High-Yield Transcript Labeling kit (Enzo). Fifteen micrograms of labeled cRNA was fragmented and sequentially hybridized to the GAPS Slides (Corning) following the manufacturer's instructions.

### Flp-In Transfection

Stable TCISM ligand (TCISML)-negative T-cell lines were established and transfected with cDNA's of TCISML candidate genes identified from microarray experiments, including: EMR2, DTR, 4-1BB, LIGHT, and CD40L. A transfection system known as the “Flp-In” method utilizing Flp-In recombinase was utilized according to the manufacturer's protocol. Eight different constructs (pcDNA5/FRT/EMR-2-04 containing EGF domains 2 and 5; pcDNA5/FRT/EMR-2-05 which contains EGF domains 1, 2 and 5; pcDNA5/FRT/EMR-02-07 which contains EGF domains 1, 2 and 5; pcDNA/FRT/DTR; pcDNA3/4-1BB; pcDNA5/FRT/LIGHT; pcDNA5/FRT/CD40L; and the pcDNA5/FRT control) were prepared using adherent 293 cells (adherent cell controls) or TCISM-negative Jurkat cells.

### Small Molecule Assay

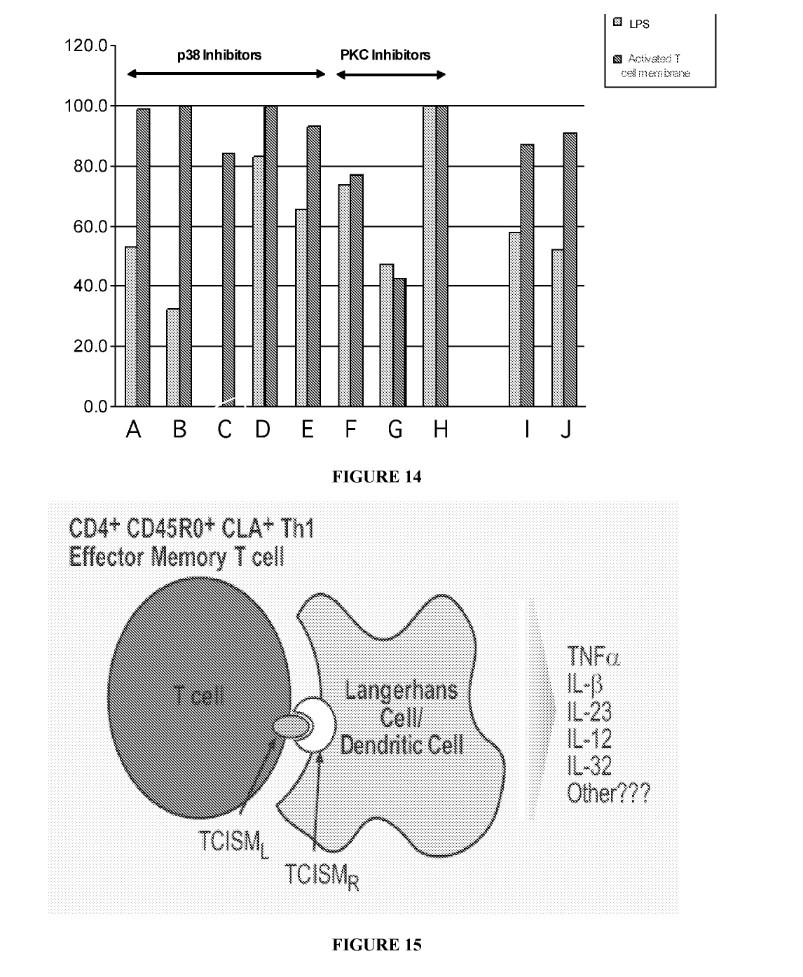

Using the T cell membrane-Mφ cell contact bioassay, small molecule antagonists were identified that differentially block anti-CD3/anti-CD28 activated T-cell mediated—but not LPS stimulated—TNFα and IL-1β production from peripheral blood resident CD14⁺ Mφ. Several kinase inhibitors were selected and assessed for the effects of these compounds in blocking TNFα and/or IL-1β production using a validated T-cell membrane-Mφ contact bioassay. It was demonstrated that Compound C, a p38 MAP kinase inhibitor, appeared to completely inhibit T-cell-mediated TNFα production from human Mφ, without having any significant effect on LPS-stimulated TNFα and IL-1β production (see FIG. 14). Other Compound C analogs either inhibited TNFα and IL-1β production from both activated T-cell membrane—and LPS-stimulated Mφ to about the same extent (about 50-100% inhibition), or showed less inhibition of cytokine production with LPS-stimulated Mφ activation (about 30-50% inhibition of LPS-activated versus about 100% inhibition of T cell-mediated cytokine production). Therefore, the activated T-cell membrane-Mt contact bioassay using human T-cells and Mφ can be used to establish high-throughput screens with recombinant TCISM (once identified and cloned) to identify orally-active, small molecule antagonists that specifically target adaptive, but not LPS-mediated, innate immunity. Some orally active, small molecule TNFα and/or IL-1β inhibitors which interfere specifically with T-cell mediated Mφ activation, leading to enhanced cytokine production but not LPS-mediated Mφ cytokine release, have a favorable therapeutic/side effect profile in T-cell mediated skin diseases such as Ps.

### Data Analysis

The statistical evaluation was performed with the statistical software “SIGMASTAT”—a tool of SIGMAPLOT V.9 (Systat Software, San Jose, Calif.). The concentration of cytokine in the group of activated H9 cells knocked out using siRNA and/or small molecule compounds and in the group of activated H9 cells was compared using the Student T test. The significance level for all comparisons was set at the common standard of 0.05.

### Statistical Considerations

By comparing the readout of our bioassay between control cells, stimulated cells, and stimulated cells where the expression of potential TCISM candidates has been knocked out using siRNA and/or small molecule compounds, one can readily validate the candidate proteins.

One of the advantages of using small molecule compounds is the ability to elucidate the p38 signaling pathway activity of TCISM as well as being orally active agents to inhibit TCISM.

### Identification of TCISM Ligand Candidates

The cell-cell contact bioassay indicated that TCISML is highly expressed on purified membranes obtained from PMA/PHA stimulated primary T-cells, Hut-78 and H9 cells, but not in Molt4, Jurkat or RAJI B-cells. Expression profiling data from activated versus non-activated cells using eighteen separate TCISML(+) versus TCISML(−) comparison conditions showed significant differences in gene expression between these different cell lines. Computational assessment indicated that TCISM molecules were up-regulated in the TCISML(+) T-cell lines and down-regulated in the TCISML(−) T-cell lines. Approximately 10,000 out of 50,000 genes resulting from microarray experiments were examined overall. TCISML candidates were determined using these criteria: (1) they are a membrane-associated; (2) they are up-regulated by more than 2-fold in stimulated Hut-78 and H9 cells, but not in Molt4, Jurkat and Raji cells; and (3) their expression levels are confirmed by qRT-PCR. Subsequent data analysis reduced the overall list of 10,000 genes to a list of just over 100 membrane-associated proteins. Log/log intensity plots identified five candidate human T-cell TCISML genes: Diphtheria Toxin Receptor, or Heparin-Binding EGF (DTR or HB-EGF), EGF module-containing Mucin-like hormone receptor 2 (EMR2; CD97), 4-1BB (TNFRSF14), OX-40 (CD134), TNF receptor Superfamily member 9 (TNFRSF9 or LIGHT; CD248), and CD40 Ligand (CD40L; TNFRSF5). FACs was used to validate the presence of these molecules on activated H9 cells. qRT-PCR was also utilized to determine if these genes were TCISM candidates.

Proteins from a membrane preparation of stimulated and unstimulated H9 cells were separated in the first dimension using an 11 cm IPG strip pH 4-6, and in the second dimension using a 10.5-14% gradient SDS-PAGE gel. The labeled spots, representing a change in expression of at least 1.5 fold (by analysis with ImageMaster), were excised, digested using trypsin, then analyzed by nanoLC/MS/MS on an Agilent Ultra high capacity ion trap.

### Gel Analysis

Imaging was performed on a Typhoon 9400 Fluorescent Scanner (GE Healthcare) at 200 pixel resolution following destaining and rinse with water. Protein spots in the stimulated and unstimulated preparations were matched using IMAGE-Master platinum II software version 5.0 (GE Healthcare) as described below.

The intensity (3-dimensional volume) of each protein spot was normalized to the total intensity of all spots detected on a gel. A detection threshold which resulted in an average of 1500 protein spots per gel was individually adjusted before comparing the gels. Change in apparent spot density between the conditions was the criterion used for excision of spots with subsequent identification by mass spectrometry. Spots of interest were excised using a OneTouch Plus Spot Picker (The Gel Company) with 1.5 mm tips.

### In-Gel Digestion

Proteins were digested in the gel spots using trypsin. Briefly, spots from at least 2 replicates were combined and destained once with 1/1 acetonitrile and 100 mM ammonium bicarbonate, then contracted with 100% acetonitrile and vacuum dried. Spots were rehydrated with 25 ng/μl trypsin and incubated overnight at 37° C. The supernatants were collected and pooled with 2 additional extracts using 1% formic acid (aqueous) with 30% acetonitrile. Pooled extracts were vacuum-concentrated to approximately 10 μL and stored at −80° C. until used.

LC/MS/MS Analysis of Trypsin Digests

Approximately 30% of the in gel-digested sample was analyzed by reverse phase nanospray LC-MS/MS (Agilent 1100 HPLC, 75 μm ID×15 cm column, Zorbax C18). Buffer A was 0.1% formic acid. Peptides were eluted from the separating column into the mass spectrometer using a gradient of increasing buffer B (90% ACN, 0.1% formic acid) at a flow rate of 300 nl/min. Spectra were collected over a m/z range of 350-1800 Da (Agilent LC/MSD Trap XCT Ultra). Three MS/MS spectra were collected for the six most abundant m/z values, then those masses were excluded from analysis for 1 min and the next six most abundant m/z values were selected for fragmentation.

### Protein Identification Using Database Searching

Proteins were identified by searching the NCBInr, and SwissProt databases using both Mascot (Matrix Science) and Spectrum Mill (Agilent) programs. For Mascot, compound lists of the resulting spectra were generated using an intensity threshold of 10,000 and a minimum of 0.2% relative abundance with grouping within 5 scans. The compound lists were exported as .mgf files and searched against databases using a taxonomy filter for human. Parameters used in the database search were as follows: monoisotopic mass, peptide mass tolerance of 2.0 Da, fragment ion mass tolerance of 0.7 Da, tryptic peptides only allowing for 2 missed cleavages, carbamidomethylation of Cys as a fixed modification and deamidation (N,Q) and acetylation (K) as variable modifications. Similar parameters were used for the SpectrumMill search. SpectrumMill protein scores above 13, with peptide scores above 10 and scored percent intensity (SPI) above 70% were the cutoff for initial hit validation. Valid protein identifications required at least two peptide matches. The molecular weight and pI values were correlated from the gel to help substantiate identifications.

### Identification of TCISM Related to Skin Inflammation

Human T-cell lymphoma H9 cells were stimulated with PMA/Ionomycin. Cells were lysed using a chaotropic lysis buffer (7M urea, 2M thiourea, 4% CHAPS) and membrane proteins were isolated using a commercially available membrane protein extraction kit. Samples (200 μg) were loaded onto an 11 cm pH 4-7 IPG strip and focused for 30,000 Vhr prior to separation by SDS page. Spots were matched and relative quantitation measured using ImageMaster software. Proteins showing greater than 2-fold change between stimulated and unstimulated samples were excised, in gel digested with trypsin, and analyzed by nanoLC/MS/MS using an Agilent Ultra ion trap. Proteins were identified using SpectrumMill and MASCOT algorithms. Western blots, siRNA knockdown, and a cell-cell contact bioassay were used to validate protein identification, quantitation, and function.

Approximately 30 protein spots from whole cell lysates or membrane fractionated samples were observed to change at least 2-fold in expression in our preliminary experiments and were identified by nLC/MS/MS. Of these, 5 potential candidates were investigated further based on their potential functions in human T cells—Annexin VI, Enolase, FKBP4, CD81, CD316 and Ezrin. Western blots of these proteins showed that the expression of FKBP4 and Ezrin were significantly increased after activation of T cells, while the expression of enolase appears to be decreased. The expression of Annexin VI, CD81 and CD316 were unchanged after PMA/Ionomycin treatment. Of particular interest were FKBP4 and Ezrin. FKBP, or FK506 binding protein, is believed to be an immunophilin with prolyl isomerase activity that functions as a protein folding chaperone for proteins containing proline residues. It also binds the immunosuppressant molecule tacrolimus (originally designated FK506), which is used to treat patients suffering from autoimmune disorders. The FBKP-tacrolimus complex inhibits calcineurin and blocks signal transduction in the T-lymphocyte transduction pathway, possibly by interfering with binding of FKBP4 to Interferon Regulatory Factor-4 (IRF-4). Ezrin is believed to be an actin-binding protein with a proline-rich region. It is believed to be regulated by phosphoinositide lipids and is believed to be a substrate for Lck tyrosine kinase. Recently, phosphorylated Ezrin was found to be responsible for increased T cell polarization, adhesion and migration in patients with SLE. It is believed that Ezrin is also one of the key proteins mediating T cell infiltration to the skin resulting in cutaneous inflammation leading to psoriasis.

The present inventors have also shown that Alefacept (Amevive; Biogen-Idec) and Efalizumab (Raptiva; Genentech), two currently available psoriasis treatments, do not inhibit T-cell-driven Mφ cytokine production in the above model system. The mechanism of these agents has been reported to selectively inactivate subpopulations of human T cells by mediating dysfunctional immune synapse (IS) formation. Without being bound by any theory, it is believed that in some instances immunophilins, including but not limited to FKBP4, in concert with Ezrin, can serve as a scaffolding protein to maintain synapse interaction and mediate effective signaling through the IS. The functional roles of these T-cell targets can be readily assessed by using siRNA knockdown and a cell-cell contact bioassay for cytokine readout using ECL.

### In Vivo Arthritis Study

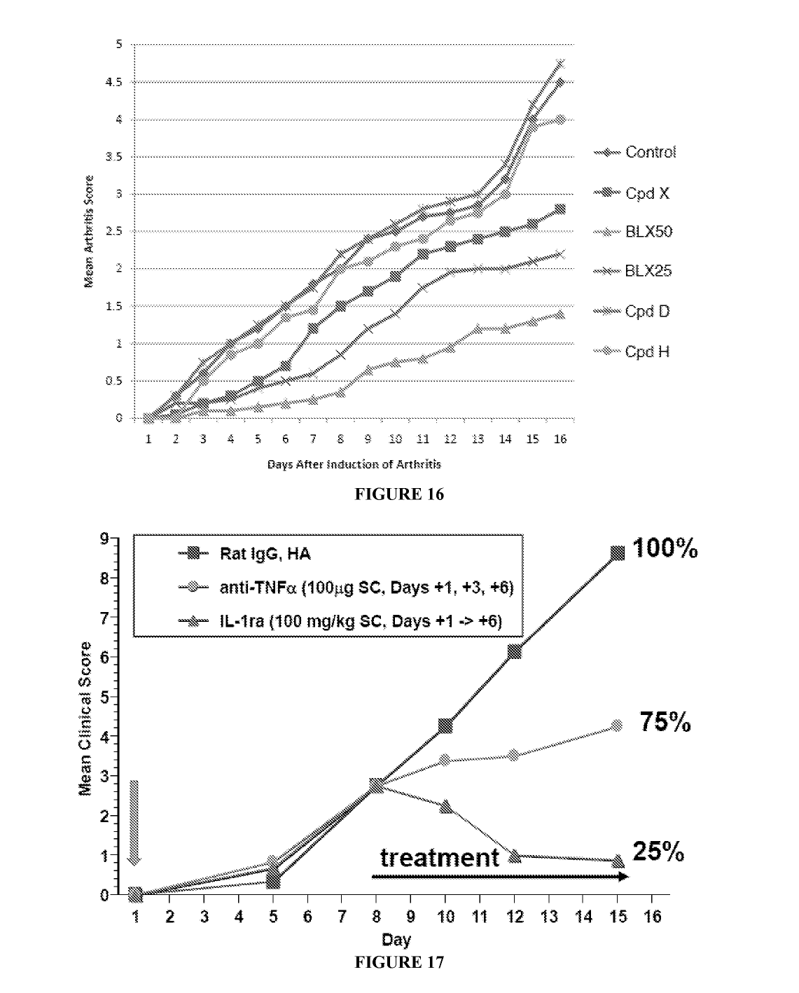

Mice were immunized with CII+CFA on day 0 and boosted on day 21 with CII+IFA. A total of 10 DBA female mice per group were used. Drugs were administered as shown and started on the first day of initial signs of paw swelling/arthritis. Each group was administered with a different TCISM modulator as shown in the Table below. Mean arthritis score (see data below) was assessed according to the procedure of Bendele et al., Arthritis & Rheumatism, 2000, 43(12), pp 2648-2659. FIG. 16 shows a graph of the mean arthritis score as a function of time for various TCISM-ligand modulators. FIG. 17 shows a graph showing control (Rat IgG, HA), anti-TNFα treated mice, and IL-1ra treated mice. In FIG. 17, a rat anti-mouse TNFα monoclonal antibody (R&D Systems) was used as a positive control to demonstrate a therapeutic effect of inhibiting TNFα or an IL-1 inhibitor known as IL-1ra (interleukin-1 receptor antagonist; Amgen). Mice were treated 8 days after showing signs of collagen induced arthritis.

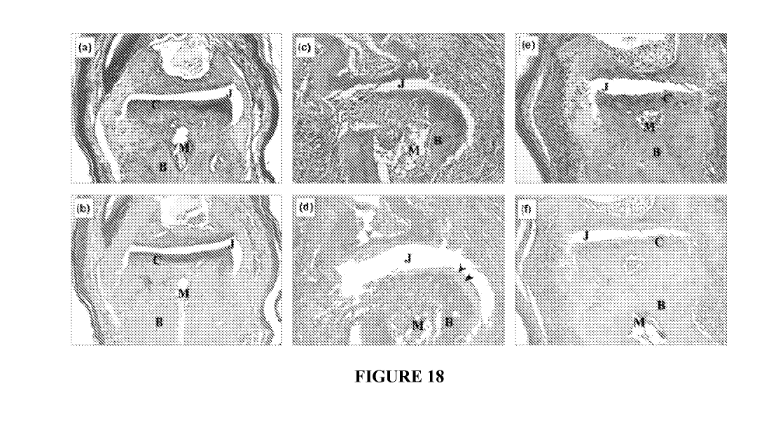

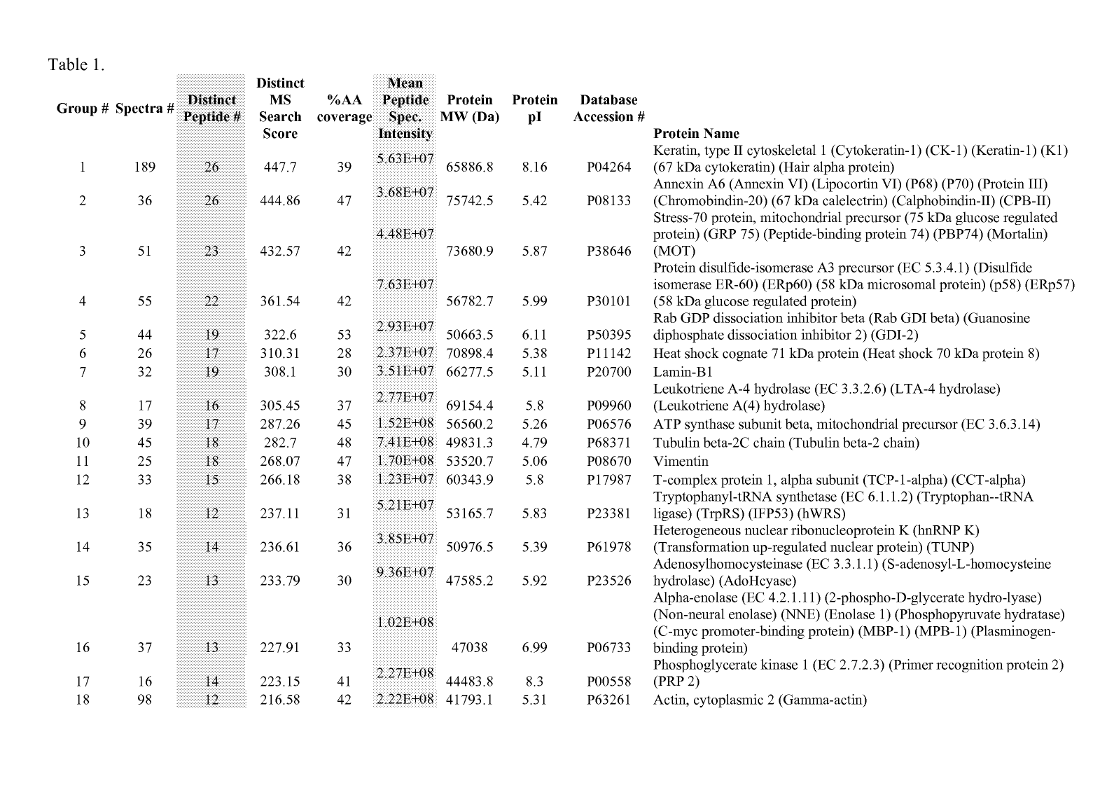

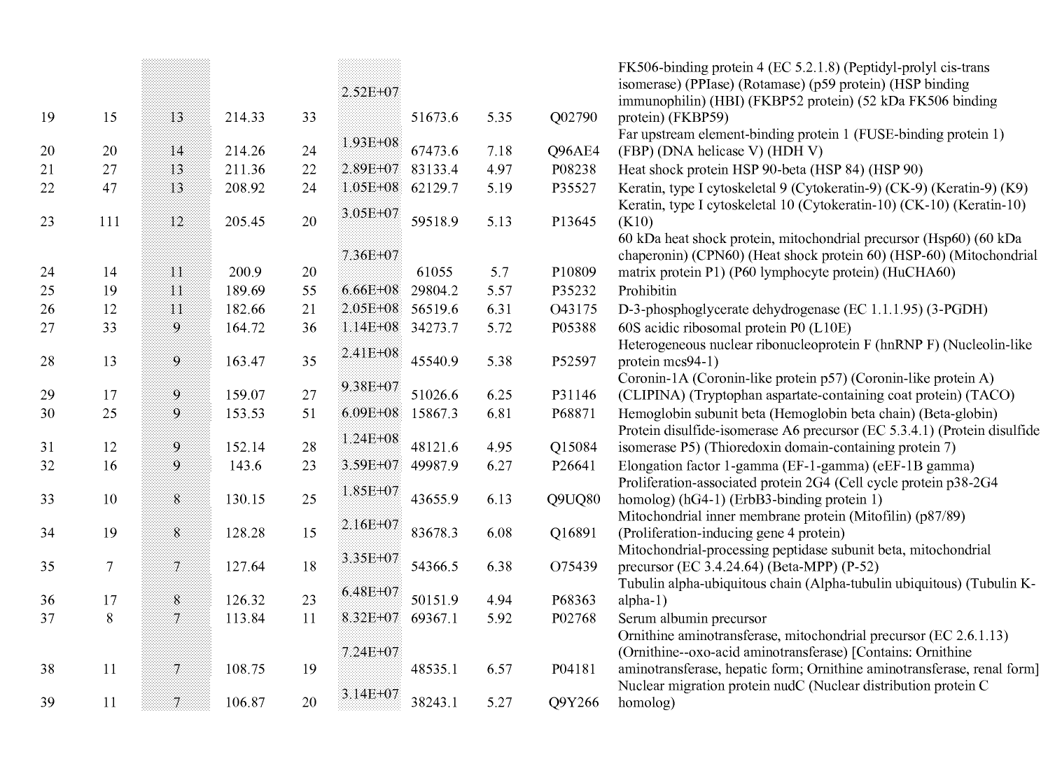

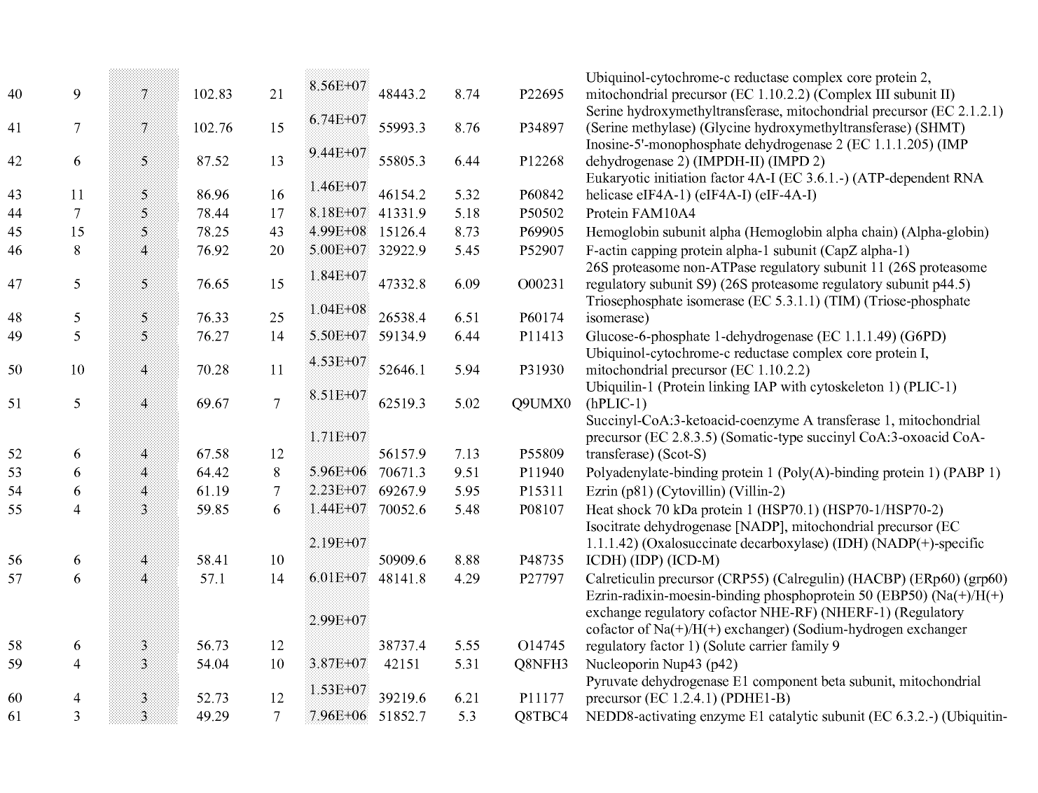

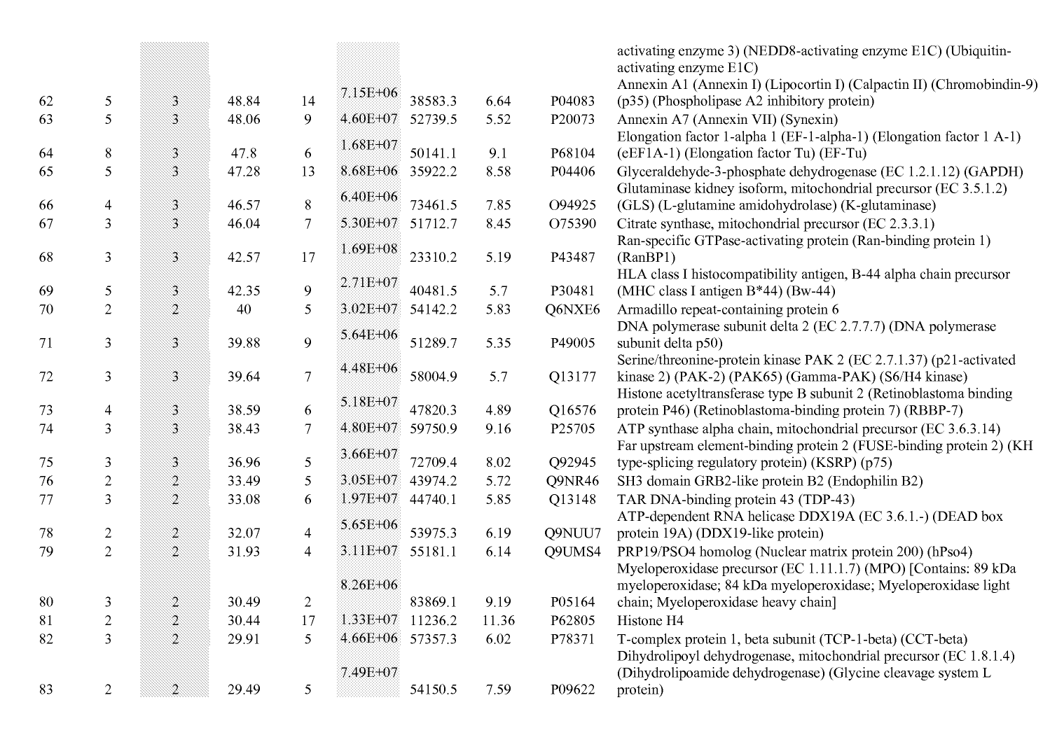

FIG. 18 is a joint histopathology of representative mice. In FIG. 18, panel A denotes a knee joint obtained from a DBA mouse with collagen-induced arthritis treated with an isotype-control rat-anti-mouse MAb (negative control) showing that no inflammation has occurred over the course of this control treatment. Panel B demonstrates that there is minimal inflammation that has occurred in the knee joint obtained from the negative control mouse. Panel C demonstrates severe inflammation, and monocytic cell and synovial cell infiltration into the knee joint obtained from a mouse treated with Compound H (50 mg/kg P.O. once per day beginning at day ⁺1) at day ⁺13 (i.e.; ⁺13 days after induction of collagen-induced arthritis). Panel D demonstrates severe inflammation and monocytic and synovial cell infiltration into the knee joint obtained from a mouse treated with Compound H (50 mg/kg P.O. once per day beginning at day ⁺1) at day ⁺13 (i.e., ⁺13 days after the induction of collagen-induced arthritis). Panel E demonstrates reduced inflammation, and virtually no monocytic cell and synovial cell infiltration into the knee joint obtained from a mouse treated with Compound C (50 mg/kg P.O. once per day beginning at day ⁺1) at day ⁺13 (i.e.; ⁺13 days after induction of collagen-induced arthritis). Panel F demonstrates reduced inflammation, virtually no monocytic and synovial cell infiltration, and preservation of cartilage and bone, obtained from a mouse treated with Compound H (50 mg/kg P.O. once per day beginning at day ⁺1) at day ⁺13 (i.e., ⁺13 days after the induction of collagen-induced arthritis).

TABLE
Assessment of different small molecule TNFα inhibitors
in Murine Collagen-Induced Arthritis

| Control | Cpd X | BLX50 | BLX25 | Cpd D | Cpd H |
|---|---|---|---|---|---|
| 0 | 0 | 0 | 0 | 0 | 0 |
| 0.3 | 0.05 | 0 | 0.2 | 0.3 | 0 |
| 0.6 | 0.2 | 0.1 | 0.2 | 0.75 | 0.5 |
| 1 | 0.3 | 0.1 | 0.25 | 1 | 0.85 |
| 1.2 | 0.5 | 0.15 | 0.4 | 1.25 | 1 |
| 1.5 | 0.7 | 0.2 | 0.5 | 1.5 | 1.35 |
| 1.8 | 1.2 | 0.25 | 0.6 | 1.75 | 1.45 |
| 2 | 1.5 | 0.35 | 0.85 | 2.2 | 2 |
| 2.4 | 1.7 | 0.65 | 1.2 | 2.4 | 2.1 |
| 2.5 | 1.9 | 0.75 | 1.4 | 2.6 | 2.3 |
| 2.7 | 2.2 | 0.8 | 1.75 | 2.8 | 2.4 |
| 2.75 | 2.3 | 0.95 | 1.95 | 2.9 | 2.65 |
| 2.85 | 2.4 | 1.2 | 2 | 3 | 2.75 |
| 3.2 | 2.5 | 1.2 | 2 | 3.4 | 3 |
| 4 | 2.6 | 1.3 | 2.1 | 4.2 | 3.9 |
| 4.5 | 2.8 | 1.4 | 2.2 | 4.75 | 4 |

Control: PBS
Cpd X: 9-[(1R,3R)-trans-cyclopentan-3-ol]adenine (Adenosine A3 Antagonist; 10 mg/kg; PO)
BLX50: BLX-WS1 (p38a MAPK Inhibitor; 50 mg/kg; PO)
BLX25: BLX-WS1 (p38a MAPK Inhibitor; 25 mg/kg; PO)
Cpd D: p38α/β MAPK Inhibitor; 50 mg/kg; PO;
Cpd H: PKC Inhibitor; 50 mg/kg; PO

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A method for modulating cytokine production in monocyte lineage-derived cells of a subject comprising administering a cytokine modulator to said subject, wherein the cytokine modulator selectively binds to a T-cell cytokine-inducing surface molecule (TCISM)-ligand of a T lymphocyte or the corresponding TCISM-receptor of a monocyte lineage-derived cell, whereby binding of the cytokine modulator to the TCISM-ligand or the TCISM-receptor modulates cytokine production in monocyte lineage-derived cells.

2. The method of claim 1, wherein the TCISM-ligand comprises at least one of the TCISM-ligand listed in Table 1.

3. The method of claim 2, wherein TCISM-ligand comprises CD81, CD21, CD316, α-Enolase, FKBP4, other members of the FKBP multigene family, or a combination thereof.

4. The method of claim 1, wherein monocyte lineage-derived cells comprise monocyte lineage-derived macrophages, antigen-presenting cells (APC), dendritic cells, Langerhans cells, Kuppfer Cells, or a combination thereof.

5. The method of claim 1, wherein the T lymphocyte is CD3⁺ T lymphocyte.

6. The method of claim 1, wherein the modulated cytokine comprises Tumor Necrosis Factor-α (TNF-α), Interleukin-1β (IL-1β), Interleukin-32 (IL-32), or a combination thereof.

7. The method of claim 1, wherein TCISM-ligand is a TCISM-ligand that is present on a CD3⁺ lymphocyte.

8. The method of claim 7, wherein TCISM-ligand comprises CD81, CD21, CD315, CD316, α-Enolase, FKBP, or a combination thereof.

9. The method of claim 1, wherein the TCISM-receptor comprises a TCISM-receptor that is present on a CD68+ antigen-presenting cell.

10. The method of claim 9, wherein the TCISM-receptor comprises a receptor for CD81, a receptor for CD21, a receptor for CD315, a receptor for CD316, a receptor for α-Enolase, a receptor for FK binding protein, or a combination thereof.

11. The method of claim 10, wherein the TCISM-receptor comprises a receptor for a TCISM-ligand that is present on CD19, CD21, CD225, CD315, CD316, C3dR, CD19, CD81, BCR, CD9, CD81, KAI1/CD82, or a combination thereof.

12. A method for treating a clinical condition mediated by acute or chronic inflammation in a subject comprising administering a cytokine modulator to said subject, wherein the cytokine modulator selectively binds to a T-cell cytokine-inducing surface molecule (TCISM)-ligand of T lymphocytes or the corresponding TCISM-receptor of monocyte lineage-derived cells, whereby modulation of cytokine production by the cytokine modulator is used to treat the clinical condition mediated by acute or chronic inflammation.

13. The method of claim 12, wherein the cytokine modulator binds selectively to a TCISM-ligand that is present on the surface of CD3⁺ lymphocytes.

14. The method of claim 12, wherein the cytokine modulator binds selectively to a TCISM-receptor that is present on the surface of a CD68⁺ monocytic cell.

15. The method of claim 12, wherein the clinical condition comprises an autoimmune disease.

16. The method of claim 15, wherein the T-lymphocyte-mediated autoimmune disease comprises Rheumatoid Arthritis, Multiple Sclerosis, Crohn's Disease, Psoriasis, Psoriatic Arthritis, Graves Disease, Autoimmune Polyendocrine Syndromes, Hereditary Proteinuria Syndrome, Type I Diabetes, Systemic Lupus Erythematosus, Primary Bilary Cirrhosis, Autoimmune Thyroiditis, Hepatitis, Acquired Immunodeficiency Disease (HIV), Graft versus Host Disease, Allograft Disease, Asthma, or a combination thereof.

17. The method of claim 12, wherein the clinical condition comprises cancer.

18. The method of claim 12, wherein the clinical condition comprises a Cutaneous T-Cell Lymphoma, HTLV-I-Associated Cutaneous T-Cell Lymphoma, HTLV-II-Associated Lymphoma, Hairy Cell Leukemia, Idiopathic CD4+ T-Lymphocytopenia, Melanoma, or a combination thereof.

19. A method for treating an autoimmune disease in a subject comprising administering a therapeutically effective amount of an antagonist to a TCISM-ligand or an antagonist to the corresponding TCISM-receptor to the subject in need of such treatment.

20. A method for modulating cytokine production in monocyte lineage-derived cells of a subject comprising administering a siRNA to said subject, wherein the siRNA inhibits transcription of a gene for a T-cell cytokine-inducing surface molecule (TCISM)-ligand of a T lymphocyte, whereby inhibition of transcription of the TCISM-ligand reduces cytokine production in the subject.
